SEQUENCE LISTING (29 sequences)

SEQ ID NO: 1 - protein, 453 aa, Mus musculus
MEEEGKKGKKPGIVSPFKRVFLKGEKSRDKKAHEKVTERRPLHTVVLALPERVEPDRLLSDYIEKEVKYLGQLTSIPGYLNPSSRTEILHFIDKAKRSHQLPGHLTQEHDAVLSLSAYNVKLAWRDGEDIILRVPIHDIAAVSYVRDDAAHLVVLKTAQDPGISPSQSLCAESSRGLSAGSLSESAVGPVEACCLVIMATESKVAAEELCSLLSQVFQIVYTESTIDFLDRAIFDGASTPTHHLSLHSDDSSTKVDMKDSYDADASTFCFPDSGDVGGLPPLPFCMQTSPHSKTVSESELSTSATELLQDYMLTLRTKLSSQEIQQFAALLHEYRNGASIHEFCISLRQLYGDSRKFLLLGLRPFIPEKDSQHFENFLETIGVKDGRGIITDSFGRHRRALSTTSTSTINGNRTTGSPDDRSAPSEGDEWDRMISDISSDIEALGCSMDQDSA

SEQ ID NO: 2 - protein, 171 aa, Mus musculus
  misc_feature: Mouse Karet PTB domain
SDYIEKEVKYLGQLTSIPGYLNPSSRTEILHFIDKAKRSHQLPGHLTQEHDAVLSLSAYNVKLAWRDGEDIILRVPIHDIAAVSYVRDDAAHLVVLKTAQDPGISPSQSLCAESSRGLSAGSLSESAVGPVEACCLVIMATESKVAAEELCSLLSQVFQIVYTESTIDFLD

SEQ ID NO: 3 - protein, 444 aa, Homo sapiens
MEEEGKKGKKPGIVSPFKRVFLKGEKSRDKKAHEKVTERRPLHTVVLSLPERVEPDRLLSDYIEKEVKYLGQLTSIPGYLNPSSRTEILHFIDNAKRAHQLPGHLTQEHDAVLSLSAYNVKLAWRDGEDIILRVPIHDIAAVSYVRDDAAHLVVLKTAQDPGISPSQSLCAESSRGLSAGSLSESAVGPVEACCLVILAAESKVAAEELCCLLGQVFQVVYTESTIDFLDRAIFDGASTPTHHLSLHSDDSSTKVDIKETYEVEASTFCFPESVDVGGASPHSKTISESELSASATELLQDYMLTLRTKLSSQEIQQFAALLHEYRNGASIHEFCINLRQLYGDSRKFLLLGLRPFIPEKDSQHFENFLETIGVKDGRGIITDSFGRHRRALSTTSSSTTNGNRATGSSDDRSAPSEGDEWDRMISDISSDIEALGCSMDQDSA

SEQ ID NO: 4 - protein, 460 aa, Homo sapiens [sequence omitted]

SEQ ID NO: 5 - protein, 171 aa, Homo sapiens
  misc_feature: Human Karet 444 amino acid variant PTB domain
SDYIEKEVKYLGQLTSIPGYLNPSSRTEILHFIDNAKRAHQLPGHLTQEHDAVLSLSAYNVKLAWRDGEDIILRVPIHDIAAVSYVRDDAAHLVVLKTAQDPGISPSQSLCAESSRGLSAGSLSESAVGPVEACCLVILAAESKVAAEELCCLLGQVFQVVYTESTIDFLD

SEQ ID NO: 6 - protein, 171 aa, Homo sapiens; identical to SEQ ID NO: 5
  misc_feature: Human Karet 460 amino acid variant PTB domain

SEQ ID NO: 7 - protein, 228 aa, Mus musculus
  misc_feature: N-terminal PTB-domain-containing, truncation mutant of the mouse Karet
MEEEGKKGKKPGIVSPFKRVFLKGEKSRDKKAHEKVTERRPLHTVVLALPERVEPDRLLSDYIEKEVKYLGQLTSIPGYLNPSSRTEILHFIDKAKRSHQLPGHLTQEHDAVLSLSAYNVKLAWRDGEDIILRVPIHDIAAVSYVRDDAAHLVVLKTAQDPGISPSQSLCAESSRGLSAGSLSESAVGPVEACCLVIMATESKVAAEELCSLLSQVFQIVYTESTIDF

SEQ ID NO: 8 - protein, 226 aa, Mus musculus
  misc_feature: A C-terminal truncation mutant of the mouse Karot corresponding to amino acid residues 228-453
FLDRAIFDGASTPTHHLSLHSDDSSTKVDMKDSYDADASTFCFPDSGDVGGLPPLPFCMQTSPHSKTVSESELSTSATELLQDYMLTLRTKLSSQEIQQFAALLHEYRNGASIHEFCISLRQLYGDSRKFLLLGLRPFIPEKDSQHFENFLETIGVKDGRGIITDSFGRHRRALSTTSTSTINGNRTTGSPDDRSAPSEGDEWDRMISDISSDIEALGCSMDQDSA

SEQ ID NO: 9 - protein, 21 aa, Mus musculus
  misc_feature: Evolutionarily conserved 21 amino acid residue juxtamembraneregion of TrkA
TLGGSSLSPTEGKGSGLQGHI

SEQ ID NO: 10 - protein, 27 aa, Artificial sequence
  A biotinylated synthetic TrkA juxtamembrane region containingpeptide
MSLHFMTLGGSSLSPTEGKGSGLQGHI

SEQ ID NO: 11 - protein, 17 aa, Artificial sequence
  Myristylation sequence
MGSSKSKPKDPSQRREF

SEQ ID NO: 12 - protein, 510 aa, Rattus norvegicus
MEYEAKKGKKGFVSPIRRLVFPKAARQAAFKSNVSRRPLHSMPLYPPDYLIDPHILLCDYLEKEVKFLGHLTWVTSSLNPSSRDELLQLLDTARQLKELPLKTTPEQDSILSLSARCLLLTWRDNEELILRIPTHEIAAASYLQDDALHLLVLKTGLGVDPVPAGMDGSPGSSGRDPGPPGAAPEKRRVGTAERRHTICSLDWRMAWGGGAGAEARAAGGGGSLERQSAGARASENHSPDAYCNLVILAVANRDAAEESCALICQVFQIIYGDQSIECVDRAGYHYRPTPKRPWLSSCSESCRTDGTFAYDADLSCCSSFNGSQDTFEACYSGASTPSFHSSHCSSIDHSSWAFEQLQDYMVTLRSKLGPPEIQQFAMLLRDYRLGLPIQDYCAGLQKLYGDRRKFLLIGMRPFIPDQDIGYFEGFLESMGIREGGILTDSFGRIKRSMSSTSASAVRSYDGAAQRPEEQTFHQLLTDITHDIEALAPDDDDVYEDVSPSGGDQVEDNYL

SEQ ID NO: 13 - protein, 465 aa, Homo sapiens
MHSSCRQRRNQNLSKEIPQTEFHTGYSMENEPGIVSPFKRVFLKGEKSRDKKAHEKVTERRPLHTVVLSLPERVEPDRLLSDYIEKEVKYLGQLTSIPGYLNPSSRTEILHFIDNAKRAHQLPGHLTQEHDAVLSLSAYNVKLAWRDGEDIILRVPIHDIAAVSYVRDDAAHLVVLKTAQDPGISPSQSLCAESSRGLSAGSLSESAVGPVEACCLVILAAESKVAAEELCCLLGQVFQVVYTESTIDFLDRAIFDGASTPTHHLSLHSDDSSTKVDIKETYEVEASTFCFPESVDVGGASPHSKTISESELSASATELLQDYMLTLRTKLSSQEIQQFAALLHEYRNGASIHEFCINLRQLYGDSRKFLLLGLRPFIPEKDSQHFENFLETIGVKDGRGIITDSFGRHRRALSTTSSSTTNGNRATGSSDDRSAPSEGDEWDRMISDISSDIEALGCSMDQDSA

SEQ ID NO: 14 - protein, 171 aa, Homo sapiens; identical to SEQ ID NO: 5
  misc_feature: Human Karet isoform 1 PTB domain

SEQ ID NO: 15 - DNA, 3554 bp, Mus musculus
cacggcccacggtggcgaaggcggctttagtagcagcatgaagcgcgtccagaccgcggaggaacgagagcgggaagctaagaaactgaggctccttgaggagcttgaagacacttggcttccttacctgacccccaaagatgatgagttctatcagcagtggcagctgaaataccctaaactggttttccgagaggccggcagcataccagaggagctgcataaggaggtccccgaggcctttctcacactgcataagcatggctgcttgtttcgggacgtggtgaggatccaaggcaaagatgtgctcaccccagtgtctcgcatcctcatcggggacccaggctgcacctacaagtacttgaacaccagactcttcacggtgccctggcccgtgaagggctgcacggtcaagtacacagaggctgagatcgccgctgcatgtcagaccttcctaaagctcaatgactacctccaggtggagaccatccaggccttggaagaactggctgtcagagagaaggccaatgaagacgctgtgccactgtgcatggcagagttccccagggccggcgtggggccgtcctgcgatgatgaagtggaccttaagagcagagcagcctacaacgtgactttgctaaacttcatggatcctcagaagatgccctacttgaaagaggagccctatttcggcatggggaagatggcggtgagctggcatcacgatgagaacctggtggacaggtcagccgtggcagtgtacagctatagctgcgaaggctctgaggatgaaagtgaggacgagtccagcttcgaaggcagagatcctgatacttggcatgttggttttaagatctcatgggacatcgagacaccaggattaacaatccctcttcaccagggagactgctatttcatgctggatgacctcaatgccacccaccagcactgtgttttggctggctcacagcctcggtttagttccactcaccgtgtggcagagtgctcaacaggcaccttggattatatcttagaacgctgtcagttggcgctgcagaatgtcctcaatgactcagacgatggcgacgtctcgttgaaatcctttgatcctgcagttttgaaacaaggagaggaaatccataatgaggtggagtttgagtggctgaggcagttctggtttcaaggcagtcgatacaaactttgcaccgattggtggtgtgagcccatgactcacctggaggggctgtggaagaagatggagagcatgacaaatgcggtgctccgtgaagttaaaagagaggggctcccggtggaacaaaggagtgagattctgtctgccatcctggtcccgctcaccgtgcgccagaacctgaggaaggagtggcatgccaggtgccagtcccgagtcgtccggactttaccagtacagcagaaaccagactgccggccatattgggagaaggatgacccttccatgcctctgccctttgacctcacagacgtggtttccgagctcagaggccagctgctggaagcaagatcctagaggaagcacaagtcttaggtggagaaggaaaagatatccttggctttcttcctcaaaccttggacaagggtagtgtagactcctctcaacccagtggttataggacctggttccaattcaaaacagctagagaacaaagcccacagattctgaagtgttgaaacgttactgtgttttagtctgacttggtgttaaattagagcacccctccctccccccaaatattcttcactcaccaccccccacacacacctgaagcccttttgtctcccaaggcagcagcagggatgggctaagcctgtgacaagcctcacctgctctcctctttggctggccacctcaaagacctggaagtaggcctggccatagggtgacagaactagagacatccatcccagcttctcaccgtggcttctagtggttagaccatccaagatggcagccagtgtctcaccgcggctgcacagatataaattgagctcagttgtcattaaaactccattcttcagttgccctggcgcatgctgatgactgctgacttagacgacacatacagatgtttatgtgttccacatctgtgagcgagccttccttctcccagccttctgtagtggctaaaacatgggggaaaacagtgaaaggcagtcaccgccgtcacctctttcctcctctgagagggaaggggccatgagccctgtgcactgcacacaggaagaggaaggagctacaaggagacagccaaaccctgtctttgcggggcttccctgatccagtgacagaggccagccctgggaccactgttctgtggagctagtgtttctcctagaattccccactcatagatactgacaggtcgtatgggggcttgaagcacccagttcccaaccccgtgacgtcagagcatcagagagagcatggactctgatttcaaagtgccaggagtaagatccaccgcagcactcactccatgacctttgcatgtccttgggtctcagttttctcatctgtatgattttgggaggtacccaaaatgccaactcctaatgtgaaaatgacgtgatatgacagagctacagatctagagtaatttctagcataccgctgaatatccgatacgttccagtgtcgttacgtgagctgacctggaagaagccgtcttcctcccccccccccccccgggtctgcagtcctttgccgtcttgtgtctcgtggcccacatctttccctagtgacctgaggccttttggtggctgaaaggttgacaacagttagcccatgattctcttgtattggccgagacctgcaattggaaggacacaacatagttgtggtttttgctgcatccaggattccagttaaggtgccttcctgcctagaatccaccatagaagctgcactccaccagccccaagggcttcctggtgcagggacccaaggctctcagaccttgatttgttcccctttgtctctgagccaggacttcctcatcagtagagagtgaggtacattctcacggggtaaaatggaacaacctaagtaacggtatagttggcttcatatgtcacagactggcccgtctccccatccccagccatctggctctgtggtttattaggccccgtgatatttctttgttctatttaattgggtgccagaattaaaaattgagatattccacataaaataaaaatgtctgcattctctttagaagccagaatacctgtgagcccctgggtctccataatcgcatggcggtggcttggcaatgcctgtgacctttgtgctctgctgtgaccctgttgctccctgtcaccattttgtaaacctgcgtccgtcccagcagcactttctagagctgcttgctcgcatccttagtagaaagcagtagcgttgagtgctcagtccctgctcctcccattggtctaagttcatggtctgcttgtattgctgtgctgtgtgcatttgttcatacacaataaagaattaaacgatgaaaaaaaaaaaaaaa

SEQ ID NO: 16 - protein, 502 aa, Mus musculus
MKRVQTAEEREREAKKLRLLEELEDTWLPYLTPKDDEFYQQWQLKYPKLVFREAGSIPEELHKEVPEAFLTLHKHGCLFRDVVRIQGKDVLTPVSRILIGDPGCTYKYLNTRLFTVPWPVKGCTVKYTEAEIAAACQTFLKLNDYLQVETIQALEELAVREKANEDAVPLCMAEFPRAGVGPSCDDEVDLKSRAAYNVTLLNFMDPQKMPYLKEEPYFGMGKMAVSWHHDENLVDRSAVAVYSYSCEGSEDESEDESSFEGRDPDTWHVGFKISWDIETPGLTIPLHQGDCYFMLDDLNATHQHCVLAGSQPRFSSTHRVAECSTGTLDYILERCQLALQNVLNDSDDGDVSLKSFDPAVLKQGEEIHNEVEFEWLRQFWFQGSRYKLCTDWWCEPMTHLEGLWKKMESMTNAVLREVKREGLPVEQRSEILSAILVPLTVRQNLRKEWHARCQSRVVRTLPVQQKPDCRPYWEKDDPSMPLPFDLTDVVSELRGQLLEARS

SEQ ID NO: 17 - DNA, 1904 bp, Homo sapiens
gcgcggccggggcggagacttcgggcccggctggcgggcggcgccgggagcgcgggggcggcgggcccgggtcgagcatgtagcggctgctggcggcggggctcccggggcgggccgggcgggccgcgggagccgcacgcggcgatatggaagaggagggcaagaagggcaagaagcctggaattgtctcgccatttaaacgagtattcctaaaaggtgaaaagagtagagataagaaagcccatgagaaggtgacagagaggcgccctctgcacactgtggtgttgtcattgcctgagcgcgtcgagccagacagactgctgagcgactatattgagaaggaggtaaagtatttaggtcagttaacgtccataccaggatacctgaatccctccagtaggactgaaatcctgcatttcatagacaatgcaaagagagcccaccagcttccgggacacttgactcaggagcacgatgctgtgctcagcctgtctgcgtacaacgtcaagctggcctggagggacggggaggatatcatcctcagggtgcccatccatgacatcgccgccgtctcctatgttcgggatgacgctgcacacctggtggtcctgaagacagcccaggacccagggatctcccccagccagagtctgtgtgcggaaagttccagaggcctcagtgcaggctccctgtcggagagtgcagttgggcccgtggaggcatgctgcctggtcatcctggctgcagagagcaaggtcgctgcggaggagctttgctgtctgctaggccaggtcttccaggttgtttacacggagtccaccatcgactttctggacagagcgatatttgatggggcctctaccccgacccaccacctgtccctgcacagcgatgactcttctacaaaagtggacattaaggagacctacgaggtggaagccagcactttctgcttccctgaatctgtggatgtgggtggtgcatcaccccacagcaagaccatcagtgagagcgagctgagcgccagcgccactgagctgctgcaggactacatgctgacgctgcgcaccaagctgtcatcacaggagatccagcagtttgcagcactgctgcacgagtaccgcaatggggcctctatccacgagttctgcatcaacctgcggcagctctacggggacagccgcaagttcctgctgcttggtctgaggcccttcatccctgagaaggacagccagcacttcgagaacttcctggagaccattggcgtgaaggatggccgcggcatcatcactgacagctttggcaggcaccggcgggccctgagcaccacatccagttccaccaccaatgggaacagggccacgggcagctctgatgaccggtcggcaccctcagagggggatgagtgggaccgcatgatctcggacatcagcagcgacattgaggcgctgggctgcagcatggaccaggactcagcatgatggacagtggatgggggggcacccacaccttccgcgcagtcgtcataggccttcccagaaggagctgcccagacctgcgtgtcagcccttggtggtggccagggagaggcgcccggtgcagatggccccgggcggcccaggtcctctactgtgaaggagcagggagctgccgagggacacgagcctcagtgcggggtggaaggctctttgccttgtccaccagggctcagccaagccctgcagtgtgtccccgctcggggagggcccggccgagcgggcagggagagccagtcctgtcggctgggcccttggacggctgtcagttttgcacatgatgttcctattgtaactctcagagaccttaaaaagaagtttactgcaatgtgaataatttaatctctggttgccaagcaaaaaaaaaaa

SEQ ID NO: 18 - DNA, 2140 bp, Homo sapiens
tttgaaattaatgtccttctgttgactgcagaatttctgctggaaatgcagggaggctgcttgtggggttcgcgtgctgtgggaagtagggaaaatgagctggtttactttgtgtctttttgtgcatgcagactgtttggattttgccaacagtttctgggtgctgcctgctttttaaacttttaaaaaattgtaaacagcttcagtggctttgcagaaggaagctgtggtttctggtcttcctgttcagtcatgtttttttcagagggagggctaacatctggccatatttttcaacaaattcagaagtgagtagagaatgcatagtagctgtcggcagaggaggaaccagaatctctccaaagaaattcctcaaacagaatttcacacagggtattccatggagaatgagcctggaattgtctcgccatttaaacgagtattcctaaaaggtgaaaagagtagagataagaaagcccatgagaaggtgacagagaggcgccctctgcacactgtggtgttgtcattgcctgagcgcgtcgagccagacagactgctgagcgactatattgagaaggaggtaaagtatttaggtcagttaacgtccataccaggatacctgaatccctccagtaggactgaaatcctgcatttcatagacaatgcaaagagagcccaccagcttccgggacacttgactcaggagcacgatgctgtgctcagcctgtctgcgtacaacgtcaagctggcctggagggacggggaggatatcatcctcagggtgcccatccatgacatcgccgccgtctcctatgttcgggatgacgctgcacacctggtggtcctgaagacagcccaggacccagggatctcccccagccagagtctgtgtgcggaaagttccagaggcctcagtgcaggctccctgtcggagagtgcagttgggcccgtggaggcatgctgcctggtcatcctggctgcagagagcaaggtcgctgcggaggagctttgctgtctgctaggccaggtcttccaggttgtttacacggagtccaccatcgactttctggacagagcgatatttgatggggcctctaccccgacccaccacctgtccctgcacagcgatgactcttctacaaaagtggacattaaggagacctacgaggtggaagccagcactttctgcttccctgaatctgtggatgtgggtggtgcatcaccccacagcaagaccatcagtgagagcgagctgagcgccagcgccactgagctgctgcaggactacatgctgacgctgcgcaccaagctgtcatcacaggagatccagcagtttgcagcactgctgcacgagtaccgcaatggggcctctatccacgagttctgcatcaacctgcggcagctctacggggacagccgcaagttcctgctgcttggtctgaggcccttcatccctgagaaggacagccagcacttcgagaacttcctggagaccattggcgtgaaggatggccgcggcatcatcactgacagctttggcaggcaccggcgggccctgagcaccacatccagttccaccaccaatgggaacagggccacgggcagctctgatgaccggtcggcaccctcagagggggatgagtgggaccgcatgatctcggacatcagcagcgacattgaggcgctgggctgcagcatggaccaggactcagcatgatggacagtggatgggggggcacccacaccttccgcgcagtcgtcataggccttcccagaaggagctgcccagacctgcgtgtcagcccttggtggtggccagggagaggcgcccggtgcagatggccccgggcggcccaggtcctctactgtgaaggagcagggagctgccgagggacacgagcctcagtgcggggtggaaggctctttgccttgtccaccagggctcagccaagccctgcagtgtgtccccgctcggggagggcccggccgagcgggcagggagagccagtcctgtcggctgggcccttggacggctgtcagttttgcacatgatgttcctattgtaactctcagagaccttaaaaagaagtttactgcaatgtgaataatttaatctctggttgccaagcaaaaaaaaaaa

SEQ ID NO: 19 - DNA, 1902 bp, Homo sapiens
gcgcggccggggcggagacttcgggcccggctggcgggcggcgccgggagcgcgggggcggcgggcccgggtcgagcatgtagcggctgctggcggcggggctcccggggcgggccgggcgggccgcgggagccgcacgcggcgatatggaagaggagggcaagaagggcaagaagcctggaattgtctcgccatttaaacgagtattcctaaaaggtgaaaagagtagagataagaaagcccatgagaaggtgacagagaggcgccctctgcacactgtggtgttgtcattgcctgagcgcgtcgagccagacagactgctgagcgactatattgagaaggaggtaaagtatttaggtcagttaacgtccataccaggatacctgaatccctccagtaggactgaaatcctgcatttcatagacaatgcaaagagagcccaccagcttccgggacacttgactcaggagcacgatgctgtgctcagcctgtctgcgtacaacgtcaagctggcctggagggacggggaggatatcatcctcagggtgcccatccatgacatcgccgccgtctcctatgttcgggatgacgctgcacacctggtggtcctgaagacagcccaggacccagggatctcccccagccagagtctgtgtgcggaaagttccagaggcctcagtgcaggctccctgtcggagagtgcagttgggcccgtggaggcatgctgcctggtcatcctggctgcagagagcaaggtcgctgcggaggagctttgctgtctgctaggccaggtcttccaggttgtttacacggagtccaccatcgactttctggacagagcgatatttgatggggcctctaccccgacccaccacctgtccctgcacagcgatgactcttctacaaaagtggacattaaggagacctacgaggtggaagccagcactttctgcttccctgaatctgtggatgtgggtggtgcatcaccccacagcaagaccatcagtgagagcgagctgagcgccagcgccactgagctgctgcaggactacatgctgacgctgcgcaccaagctgtcatcacaggagatccagcagtttgcagcactgctgcacgagtaccgcaatggggcctctatccacgagttctgcatcaacctgcggcagctctacggggacagccgcaagttcctgctgcttggtctgaggcccttcatccctgagaaggacagccagcacttcgagaacttcctggagaccattggcgtgaaggatggccgcggcatcatcactgacagctttggcaggcaccggcgggccctgagcaccacatccagttccaccaccaatgggaacagggccacgggcagctctgatgaccggtcggcaccctcagagggggatgagtgggaccgcatgatctcggacatcagcagcgacattgaggcgctgggctgcagcatggcccaggactcagcattatggacagtggatgggggggcacccacaccttccgcgcagttgtcataggccttcccaaaaggagctgcccagacctgggtgtcagcccttggtggtggccagggaaaggcgcccggtgcaaatggccccgggcggcccaggtcctttactgtgaaggagcagggagctgccgagggacacgagcctcagtgcggggtggaaggctttttgccttgtccaccaggggtcagccaagccctgcagtgtgtccccgctcggggagggcccggccgagcgggcagggaaaaccagtcctgtcggctgggcccttggacggctgtcagttttgcacatgatgttcctattgtaactctcagagaccttaaaaaaaagtttactgcaatgttaaaaaaaaaaaaaaaaaaaaaaaaaaaaaaaaaaa

SEQ ID NO: 20 - DNA, 4294 bp, Homo sapiens
ctacgctcttccagctgtcggacctgggaaattctcctgtgctaaatcccgtggcgctcgcgggtgtcgccgcggtgcatcctgggagttgtagttttttctactcagagggagaatagctccagacgggagcaggacgctgagagaactacatgcaggaggcggggtccagggcgagggatctacgcagcttgcggtggcgaaggcggctttagtggcagcatgaagcgcaccccgactgccgaggaacgagagcgcgaagctaagaaactgaggcttcttgaagagcttgaagacacttggctcccttatctgacccccaaagatgatgaattctatcagcagtggcagctgaaatatcctaaactaattctccgagaagccagcagtgtatctgaggagctccataaagaggttcaagaagcctttctcacactgcacaagcatggctgcttatttcgggacctggttaggatccaaggcaaagatctgctcactccggtatctcgcatcctcattggtaatccaggctgcacctacaagtacctgaacaccaggctctttacggtcccctggccagtgaaagggtctaatataaaacacaccgaggctgaaatagccgctgcttgtgagaccttcctcaagctcaatgactacctgcagatagaaaccatccaggctttggaagaacttgctgccaaagagaaggctaatgaggatgctgtgccattgtgtatgtctgcagatttccccagggttgggatgggttcatcctacaacggacaagatgaagtggacattaagagcagagcagcatacaacgtaactttgctgaatttcatggatcctcagaaaatgccatacctgaaagaggaaccttattttggcatggggaaaatggcagtgagctggcatcatgatgaaaatctggtggacaggtcagcggtggcagtgtacagttatagctgtgaaggccctgaagaggaaagtgaggatgactctcatctcgaaggcagggatcctgatatttggcatgttggttttaagatctcatgggacatagagacacctggtttggcgataccccttcaccaaggagactgctatttcatgcttgatgatctcaatgccacccaccaacactgtgttttggccggttcacaacctcggtttagttccacccaccgagtggcagagtgctcaacaggaaccttggattatattttacaacgctgtcagttggctctgcagaatgtctgtgacgatgtggacaatgatgatgtctctttgaaatcctttgagcctgcagttttgaaacaaggagaagaaattcataatgaggtcgagtttgagtggctgaggcagttttggtttcaaggcaatcgatacagaaagtgcactgactggtggtgtcaacccatggctcaactggaagcactgtggaagaagatggagggtgtgacaaatgctgtgcttcatgaagttaaaagagaggggctccccgtggaacaaaggaatgaaatcttgactgccatccttgcctcgctcactgcacgccagaacctgaggagagaatggcatgccaggtgccagtcacgaattgcccgaacattacctgctgatcagaagccagaatgtcggccatactgggaaaaggatgatgcttcgatgcctctgccgtttgacctcacagacatcgtttcagaactcagaggtcagcttctggaagcaaaaccctagaaggagcacaagtctcaggcggaggagaaaaagagatcggcttttctcctccaacgttgtcatgggcttaagcaagagcagtggagacttctcttggcccctagattgtagcacccgggtcccaatccaaaacagctaggaaatggtgcccatgaagttttaaatgttttaaaatgaccctgtgttatagtctgatttggtgttaaacaggaccttcttcccccaaaattgttcagattataaaatgtgagccattcagcccccaaggtccagggcaggcgacaggaacgagcccagcgtgtgacaaagcctaacctactttcctctttcccaagctttttcagagactctggagtggacccagccctctggggaaagacagaacttagagacatcccagttactcaccacacccatagtgctgtccaatatggtagccactagctagctgtggctacttcaatttaaattcagttttaattttaattaaaaatgcagctcttcagtcgccctggccacatttcaagtgcttaacagcctcatgtggctagtgactgctgtattggacggtacagatatggaacattttcatcatcgaagaaagtcctattggacaacacttctataaaaagtttgagagcaggaattctcatttccattcgtctgtagcttctatccccaaaggcaaagaaactaaaagagaaatgactcattgaagattggcctctttcctttctctaagacaaacctaagtaaaagcctgagctttgagtcctatgctcagcacacgggaaggagatgttaataattaaaataaagttgatatcctgtctttagggagttcccttgatctcttgaaagagacacagccccatttacattatttcgtggatttcaccagcatagtatagtttttttctgtaagtccctcattcttatgtaataacaggtggaactgaggtttgaagaacctcagtggcccatcctgatgacattggagactcaaagagacaagagagagtagggtttaaaacctgagctttaagactcccactagcttcgtgtcctttggcatgttaacgtgcctcagtttcctcatctgtataatggggatatatgaaaggcaccagtcctaaggtgaacattaagtgagatgattctagttacagacttagaacaatttccagcacatagttaaatatccaggaaattctggtactgttatgtgtgggtgagctgacctggatgtagatgttttcctctctcttgctgacccctccgccagttttgtcttgtgatgccattaacacatctctccctttctgacctggctcctgcccattggtgtcccaagaaatcgtgagaatagttagccccccgtctccccagcctgttgctttctcgtgtagttgttcacagtagttgagaagttgaagagcttttgcctattgaaggtgcactgagaataaactctttcctgccaccagaattgcagtggttcacggcctgcactcattcccatgaatgcagttaatagccacagaaatgtcacattaagcaaagcagccagggtctcatcgtgttgagactcgagtctctcagaccttggattcattccctggtgtctttgagcctcagtttcctcattggtaaaagagaagtgaagcagtgtctcacagggtcattacagagattaaatgaaataaatgaaataacatagaccaggagggcgtggtgtttaaaagtcacagatggggcaccctcgggccatccagcccagtgttttctttagcccctatgatgttcattttttgttatatcccattaggtgcccatatttaaaaattgggagatttcacataaaattaaaaggtctgcattttcttttttcttttctttttttttttttttgagacacagtctcactctgtcaccaggctagagtgcagtggcacgatctcagctcactgcaacctctgcctcccaggttcaagtaattctcctgcctcagcctcccaagtagctgggactacaggcacgtgccaccacgcccagctaatttttgtatttttagcagagatggggtttcaccacattggccaggatggtctcgatctcaacctcgtgatccacccacctcggtctcccaaagcgctgggattacaggcgtgagccaccgcgccaagccaaggtctgcatttttctttagaactcagaacacccaatagtcctaggcccccatcctcgcatggcagcaagctaaataagcatcttcccactgcgagttggggcatgacccagcctatggtttgccatactccctctttttctccgttttttcattaattgtgaacctgacctgcatcaccctttcatgtcagtgctctccaaacctgcttgcttgcacccctctagtcgaaatattttgtgcttaccccaatatatgtgtgtgactattgaactctattcgtagactgcttgtactaatgtcatttgcatcataaaatattcatatccaataaacatattaaaaggatgagataagaaaccgaga

SEQ ID NO: 21 - protein, 505 aa, Homo sapiens
MKRTPTAEEREREAKKLRLLEELEDTWLPYLTPKDDEFYQQWQLKYPKLILREASSVSEELHKEVQEAFLTLHKHGCLFRDLVRIQGKDLLTPVSRILIGNPGCTYKYLNTRLFTVPWPVKGSNIKHTEAEIAAACETFLKLNDYLQIETIQALEELAAKEKANEDAVPLCMSADFPRVGMGSSYNGQDEVDIKSRAAYNVTLLNFMDPQKMPYLKEEPYFGMGKMAVSWHHDENLVDRSAVAVYSYSCEGPEEESEDDSHLEGRDPDIWHVGFKISWDIETPGLAIPLHQGDCYFMLDDLNATHQHCVLAGSQPRFSSTHRVAECSTGTLDYILQRCQLALQNVCDDVDNDDVSLKSFEPAVLKQGEEIHNEVEFEWLRQFWFQGNRYRKCTDWWCQPMAQLEALWKKMEGVTNAVLHEVKREGLPVEQRNEILTAILASLTARQNLRREWHARCQSRIARTLPADQKPECRPYWEKDDASMPLPFDLTDIVSELRGQLLEAKP

SEQ ID NO: 22 - protein, 1210 aa, Homo sapiens [sequence omitted]

SEQ ID NO: 23 - DNA, 5616 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 24 - protein, 628 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGS

SEQ ID NO: 25 - DNA, 2239 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacgggtcctaataaatcttcactgtctgactttagtctcccactaaaactgcatttcctttctacaatttcaatttctccctttgcttcaaataaagtcctgacactattcatttga

SEQ ID NO: 26 - protein, 405 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGLS

SEQ ID NO: 27 - DNA, 1595 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacaggtttgagctgaattatcacatgaatataaatgggaaatcagtgttttagagagagaacttttcgacatatttcctgttcccttggaataaaaacatttcttctgaaattttaccgttaaaaaaaaaaaaaaaaaaaaaaaaa

SEQ ID NO: 28 - protein, 705 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGPGNESLKAMLFCLFKLSSCNQSNDGSVSHQSGSPAAQESCLGWIPSLLPSEFQLGWGGCSHLHAWPSASVIITASSCH

SEQ ID NO: 29 - DNA, 2865 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacgggccaggaaatgagagtctcaaagccatgttattctgcctttttaaactatcatcctgtaatcaaagtaatgatggcagcgtgtcccaccagagcgggagcccagctgctcaggagtcatgcttaggatggatcccttctcttctgccgtcagagtttcagctgggttggggtggatgcagccacctccatgcctggccttctgcatctgtgatcatcacggcctcctcctgccactgagcctcatgccttcacgtgtctgttccccccgcttttcctttctgccacccctgcacgtgggccgccaggttcccaagagtatcctacccatttccttccttccactccctttgccagtgcctctcaccccaactagtagctaaccatcacccccaggactgacctcttcctcctcgctgccagatgattgttcaaagcacagaatttgtcagaaacctgcagggactccatgctgccagccttctccgtaattagcatggccccagtccatgcttctagccttggttccttctgcccctctgtttgaaattctagagccagctgtgggacaattatctgtgtcaaaagccagatgtgaaaacatctcaataacaaactggctgctttgttcaatgctagaacaacgcctgtcacagagtagaaactcaaaaatatttgctgagtgaatgaacaaatgaataaatgcataataaataattaaccaccaatccaacatccaga

(19) United States
(12) Patent Application Publication
(10) Pub. No.: US 2009/0036380 A1
(43) Pub. Date: Feb. 5, 2009

(54) Composition And Method For Brain Tumor Therapy

(75) Inventors: Michael Fainzilber, Rechovot (IL); Marianna Tcherpakov, Petach-Tikva (IL); Zehava Levy, Yahud (IL); Liraz Harel, Petach-Tikva (IL)

Correspondence Address: MARTIN D. MOYNIHAN d/b/a PRTSI, INC., P.O. BOX 16446, ARLINGTON, VA, 22215, US

(73) Assignee: Yeda Research and Development Co., Ltd., Rechovot (IL)

(21) Appl. No.: 12/224,999

(22) PCT Filed: Mar. 14, 2007
(86) PCT No.: PCT/IL07/00334

§ 371 Date: Sep. 12, 2008

Related U.S. Application Data
(60) Provisional application No. 60/781,724, filed on Mar. 14, 2006.

(51) Int. Cl. A61K 38/16; A61P 35/00

(52) U.S. Cl. 514/12

(57) ABSTRACT

A method of treating a disease associated with abnormal apoptosis in a subject in need thereof is disclosed. The method comprises administering to the subject a therapeutically effective amount of at least one agent capable of increasing and/or stabilizing an interaction between at least an active portion of TrkA and at least an active portion of Karet, thereby treating the disease in the subject.

### FIELD AND BACKGROUND OF THE INVENTION

The present invention relates to methods of treating diseases associated with abnormal apoptosis. More particularly, the present invention relates to methods of treating cancers, such as cancers of neural origin, via induction of apoptosis of cancer cells.

Cancers of neural origin comprise various types of malignancies, such as neuroblastomas and medulloblastomas, for which no satisfactory/optimal therapy is available, and which are associated with significant morbidity and mortality.

Neuroblastoma is a neural crest-derived tumor of the sympathetic nervous system, and is one of the most common pediatric solid tumors. Most infants diagnosed with neuroblastoma before one year of age spontaneously undergo complete regression of the disease, whereas older children frequently have metastatic disease that is extremely difficult to reverse with current modalities of chemotherapy (Nat Rev Cancer, 2003. 3 (3): p. 203-16).

Medulloblastoma is a highly malignant and poorly understood tumor of the cerebellum, categorized as central neuronal/neuroblastic tumors (Katsetos, C. D., et al. Acta Neuropathol (Berl), 2003. 105 (1): p. 1-13; Ullrich, N. J. and S. L. Pomeroy, Neurol Clin, 2003. 21 (4): p. 897-913; Pomeroy, S. L. and L. M. Sturla. Pediatr Neurosurg, 2003. 39 (6): p. 299-304), which accounts for approximately 20 percent of primary brain tumors in children.

The nerve growth factor (NGF) family of neurotrophins has diverse roles in the nervous system, including regulation of progenitor cell numbers, modulation of neurite outgrowth and growth cone guidance, and control of death or survival of neurons and other cell types. These different activities are mediated by two distinct classes of cell surface receptors, the trk family of receptor tyrosine kinases (RTKs; Huang and Reichardt, 2003; Segal, 2003), and the p75 neurotrophin receptor, which belongs to the tumor necrosis factor (TNF) receptor superfamily (Roux and Barker, 2002). The tropomyosin-related kinase (Trk) family encodes the specific receptor tyrosine kinases (RTKs) for members of the neurotrophin family. Trk receptor signaling and activities have been well characterized over the past 15 years, and it is now widely accepted that a primary role of trk receptors is the control of neuronal survival in response to limiting amounts of neurotrophin ligands, and neuronal proliferation and differentiation (Huang and Reichardt, 2003). The p75 receptor, on the other hand, has been implicated in diverse neuronal responses, including increased differentiation or survival, inhibition of regeneration, and initiation of apoptotic cell death (Barker, 2004; Bronfman and Fainzilber, 2004). In recent years studies have been performed aimed at elucidating the mechanisms and signaling pathways underlying the diversity of p75 functions, using the Ras Rescue System (RRS) to screen for interactors of the p75 intracellular domain. Such studies have identified the MAGE protein necdin as an interactor for the intracellular domain of p75, and demonstrated that the interaction is enhanced by ligand stimulation (Tcherpakov et al., 2002). In these studies PC12 cells transfected with necdin were shown to exhibit accelerated differentiation in response to NGF. It was hypothesized that the p75-necdin mediated signal must synergize with a TrkA signaling pathway, since effects of necdin on differentiation were ligand-dependent and not seen in NNR5 cells, a PC12-derived line that lacks TrkA, or upon application of BDNF to PC12 cells. Studies employing an antisense approach on embryonic DRG cultures also supported a differentiation or survival-promoting role for necdin in NGF-responsive neurons (Takazaki et al., 2002). It was hypothesized that this could be due to a change in trk-p75 association resulting in ‘freeing’ TrkA from some inhibitory constraint imposed by p75, or could be due to an independent signal emanating from p75 and transduced by necdin. The propensity of necdin for homo-oligomerization and its cytoplasmic localization in transfected PC12 cells both supported the likelihood of necdin acting as a cytoplasmic adaptor for a p75-induced signaling complex (Tcherpakov et al., 2002).

TrkA-dependent cell death is an atypical phenomenon that has been described in very few reports in the literature, primarily in pediatric tumor cells of neural origin like neuroblastoma and medulloblastoma (Ohira, M., et al. Cancer Lett, 2005. 228 (1-2): p. 5-11; Bono, F., I. Lamarche, and J. M. Herbert. FEBS Lett, 1997. 416 (3): p. 243-6; Chou, T. T. et al. J Biol Chem, 2000. 275 (1): p. 565-70; Lucarelli, E., D. et al. Eur J Cancer, 1997. 33 (12): p. 2068-70; Muragaki, Y., et al. J Neurosci, 1997. 17 (2): p. 530-42; Rubin, J. B. and R. A. Segal. Cancer Treat Res, 2003. 115: p. 1-18; Woo, C. W. et al. Oncogene, 2004. 23 (8): p. 1522-30; Yan, C., et al. Cancer Res, 2002. 62 (17): p. 4867-75).

Expression of TrkA in neuroblastoma cells is correlated with good prognosis and is more likely to be found in the younger patients (Brodeur, Nat Rev Cancer, 2003. 3 (3): p. 203-16; Schramm, A., et al. Cancer Lett, 2005. 228 (1-2): p. 143-53; Nakagawara, A. Cancer Lett, 2001. 169 (2): p. 107-14; Nakagawara, A. and M. Ohira. Cancer Lett, 2004. 204 (2): p. 213-24) NGF activation of TrkA-transfected neuroblastoma was shown to decrease cell number via a MAPK pathway, which caused decrease in N-myc (Woo, C. W. et al. Oncogene, 2004. 23 (8): p. 1522-30), while recently another study showed TrkA induced apoptosis through a p53, Bcl-2 and caspase-3 dependent mechanism (Lavoie, J. F., et al. J Biol Chem, 2005. 280 (32): p. 29199-207). Neuroblastoma is considered to be an aberration in normal neural crest development, therefore neuronal cell death through TrkA may occur in early development of the nervous system (Nakagawara, A. and M. Cancer Lett, 2004. 204 (2): p. 213-24).

Nodular/desmoplastic medulloblastomas are a histopathological subtype containing “islands” of cells with round nuclei and abundant cytoplasm. Localized expression of TrkA or TrkC has been detected in such islands, in parallel with concentrations of apoptotic or differentiating cells (Eberhart, C. G., et al. J Neuropathol Exp Neurol, 2001. 60 (5): p. 462-9). These observations, together with the fact that TrkC is a positive prognostic indicator in medulloblastoma (Grotzer et al., 2000; Pomeroy et al., 1997) suggest that neurotrophin signaling may be involved in apoptosis or neuronal differentiation of tumors with a positive clinical outcome. A similar situation may occur in neuroblastomas, which are derived from primitive cells of the sympathetic nervous system.

Spontaneous regression or differentiation of medulloblastoma or neuroblastoma may parallel normal developmental processes in the progenitor cell populations (Kim et al., 2003; Nakagawara and Ohira, 2004; Pomeroy et al., 1997). Taken together with the in-vitro data from trk transfected medulloblastoma cells (Chou et al., 2000; Muragaki et al., 1997), the emerging picture is that specific and unusual trk-dependent cell death or differentiation pathways can be activated in pediatric tumors of neural origin.

Although TrkC is a positive prognostic indicator in medulloblastoma (Pomeroy, S. L., et al. J Neurooncol, 1997. 35 (3): p. 347-52; Grotzer, M. A., et al. Klin Padiatr, 2000. 212 (4): p. 196-9; Kim, J. Y., et al. Cancer Res, 1999. 59 (3): p. 711-9), NGF treatment of human medulloblastoma cells (MED 283) stably transfected to express TrkA using a retroviral expression vector was shown to cause significant levels of cell death that were not observed in wild-type uninfected cells, cells expressing an empty vector, or cells expressing the TrkC receptor (Muragaki et al., 1997). Furthermore, medulloblastoma cells engineered to express p75, a member of the tumor necrosis factor receptor superfamily, also did not undergo apoptosis. NGF-induced apoptosis in TrkA-medulloblastoma cells was inhibited by addition of the kinase inhibitor K-252A, or by mutation of the ATP-binding site or both tyrosines 490 and 785 in the receptor (Chou, T. T. et al. J Biol Chem, 2000. 275 (1): p. 565-70; Muragaki, Y., et al. J Neurosci, 1997. 17 (2): p. 530-42; Chou, T. T. et al. J Biol Chem, 2001. 276 (44): p. 41120-7). The specific interactors mediating this intriguing effect have not been identified to date.

Thus, in view of trk's role in the regulation of cell death in diseases such as pediatric tumors of neural origin, a potentially optimal strategy for treating such diseases would be to identify executor molecules involved in regulation of trk-dependent cell death, and to suitably regulate the levels/activity of such molecules so as to induce therapeutic cellular apoptosis.

Thus, several prior art approaches have been employed or suggested in order to treat pediatric tumors of neural origin via induction of trk-mediated cell death.

One approach proposes treating medulloblastoma by retrovirally transducing medulloblastoma tumor cells to express TrkA, optionally in combination with administration of nerve growth factor (NGF), on the basis of in-vitro studies utilizing immortalized cell lines (Muragaki Y. et al., 1997).

Another approach proposes treating glioma by transfecting glioma tumor cells to express trkA, optionally in combination with administration of NGF, on the basis of in-vitro studies utilizing immortalized cell lines (Kokunai T. et al., 1999. J. Neurooncol. 42:23-34).

Yet another approach proposes treating neuroblastoma or medulloblastoma tumors via delivery of the Trk inhibitor CEP-751 to neuroblastoma or medulloblastoma tumor cells, optionally in conjunction with transfection of tumor cells to express TrkB, based on tumor xenograft models generated using immortalized cell lines (Evans A E. et al., 1999. Clin Cancer Res. 5:3594-602).

Still another approach proposes treating medulloblastoma using regulation of putative novel Ras and/or Raf signaling pathways, on the basis of studies employing biochemical dissection of NGF/TrkA-mediated apoptotic pathways in immortalized cell lines (Chou et al., 2000).

None of the aforementioned approaches, however, have been attempted in a subject having an endogenous tumor, nor in any human patient. Consequently, none of the aforementioned prior art approaches has succeeded in demonstrating treatment of any disease in a human patient.

Thus, all prior art approaches have failed to provide an adequate solution for treatment of diseases, such as pediatric tumors of neural origin, via upregulation of trk-mediated apoptosis.

There is thus a widely recognized need for, and it would be highly advantageous to have, a method devoid of the above limitation.

### SUMMARY OF THE INVENTION

The present invention discloses the use of regulation of TrkA-Karet interaction for inducing death of pathologically hyperproliferating cells, such as neurological tumor cells. This use can be effected in a variety of ways as further described and exemplified hereinbelow.

According to one aspect of the present invention there is provided a method of treating a disease associated with abnormal apoptosis in a subject in need thereof, the method comprising administering to the subject a therapeutically effective amount of at least one agent capable of modulating an interaction between TrkA or EGFR and CCM2, thereby treating the disease in the subject.

According to another aspect of the present invention there is provided a method of treating a cancer in a subject in need thereof, the method comprising administering to the subject a therapeutically effective amount of Fatso in cells of the cancer of the subject, thereby treating the cancer in the subject.

According to yet another aspect of the present invention there is provided use of Fatso for the manufacture of a medicament identified for treating cancer.

According to still another aspect of the present invention there is provided use of CCM2 for the manufacture of a medicament identified for treating cancer.

According to an additional aspect of the present invention there is provided a pharmaceutical composition comprising as an active ingredient a therapeutically effective amount of CCM2, and a pharmaceutically acceptable carrier.

According to yet an additional aspect of the present invention there is provided a pharmaceutical composition comprising as an active ingredient a therapeutically effective amount of Fatso, and a pharmaceutically acceptable carrier.

According to still further features in the described preferred embodiments the TrkA is a polypeptide which comprises SEQ ID NO:9 or 10.

According to still further features in the described preferred embodiments the CCM2 is capable of interacting with the TrkA or the EGFR in a pathological cell associated with the disease, and of inducing death of the pathological cell.

According to still further features in the described preferred embodiments the disease is characterized by pathological cells expressing the TrkA and/or the EGFR.

According to still further features in the described preferred embodiments modulating the interaction is increasing and/or stabilizing the interaction between the TrkA or the EGFR and the CCM2.

According to still further features in the described preferred embodiments the disease associated with abnormal apoptosis is a cancer.

According to still further features in the described preferred embodiments the cancer is of neural origin.

According to still further features in the described preferred embodiments the cancer is a pediatric cancer.

According to still further features in the described preferred embodiments the cancer is a brain tumor.

According to still further features in the described preferred embodiments the cancer is a medulloblastoma, a neuroblastoma or a pheochromocytoma.

According to still further features in the described preferred embodiments the cancer is a neck cancer.

According to still further features in the described preferred embodiments the cancer is a lung cancer.

According to still further features in the described preferred embodiments the at least one agent comprises CCM2.

According to still further features in the described preferred embodiments the CCM2 comprises an amino acid sequence selected from the group consisting of SEQ ID NOs:1, 3, 4, 12 and 13.

According to still further features in the described preferred embodiments the at least one agent comprises nerve growth factor.

According to still further features in the described preferred embodiments the at least one agent comprises TrkA.

According to still further features in the described preferred embodiments the administration of the therapeutically effective amount of the at least one agent is effected by gene therapy.

According to still further features in the described preferred embodiments the Fatso comprises a polypeptide having an amino acid sequence as set forth by SEQ ID NO:16 or 21.

According to still an additional aspect of the present invention there is provided a method of treating a cancer in a subject in need thereof, the method comprising administering to the subject a therapeutically effective amount of at least one agent capable of increasing and/or stabilizing an interaction between at least an active portion of TrkA and at least an active portion of Karet, thereby treating the cancer in the subject.

According to further features in preferred embodiments of the invention described below, the active portion of Karet is capable of interacting with TrkA in a cancer cell associated with the cancer, and of inducing death of the cancer cell.

According to another aspect of the present invention there is provided a method of treating a disease associated with abnormal apoptosis in a subject in need thereof, the method comprising administering to the subject a therapeutically effective amount of at least one agent capable of modulating an interaction between at least an active portion of TrkA and at least an active portion of Karet, thereby treating the disease in the subject.

According to still further features in the described preferred embodiments, the at least one agent further comprises nerve growth factor.

According to still further features in the described preferred embodiments, the at least one agent further comprises TrkA.

According to still further features in the described preferred embodiments, the at least one agent comprises the active portion of Karet.

According to still further features in the described preferred embodiments, administering the therapeutically effective amount of the at least one agent comprises administering to the subject a nucleic acid construct which comprises a nucleic acid sequence encoding a polypeptide which comprises an amino acid sequence encoding TrkA.

According to still further features in the described preferred embodiments, administering the therapeutically effective amount of the at least one agent comprises administering to the subject a nucleic acid construct which comprises a nucleic acid sequence encoding a polypeptide which comprises an amino acid sequence having at least 70 percent homology to the amino acid sequence set forth by SEQ ID NO: 7 or 1.

According to still further features in the described preferred embodiments, the nucleic acid construct further comprises one or more transcription control sequences being capable of driving expression of the nucleic acid sequence in cancer cells of the cancer.

According to still further features in the described preferred embodiments, the active portion of Karet is capable of interacting with TrkA in a pathological cell associated with the disease, and of inducing death of the pathological cell.

According to still further features in the described preferred embodiments, modulating the interaction between the at least an active portion of TrkA and the at least an active portion of Karet is increasing and/or stabilizing the interaction between the at least an active portion of TrkA and the at least an active portion of Karet.

According to still further features in the described preferred embodiments, the active portion of Karet is a polypeptide having an amino acid sequence which has at least 70 percent homology to the amino acid sequence set forth by SEQ ID NO: 7.

According to still further features in the described preferred embodiments, the active portion of Karet is a polypeptide having an amino acid sequence which has at least 70 percent homology to the amino acid sequence set forth by SEQ ID NO: 1.

According to still further features in the described preferred embodiments, the active portion of TrkA is a polypeptide having an amino acid sequence which has at least 70 percent homology to the amino acid sequence set forth by SEQ ID NO: 9.

According to still further features in the described preferred embodiments, the active portion of TrkA is a polypeptide having an amino acid sequence which has at least 70 percent homology to the amino acid sequence set forth by SEQ ID NO: 10.

According to still further features in the described preferred embodiments, the disease associated with abnormal apoptosis is a cancer.

According to still further features in the described preferred embodiments, the cancer is a cancer which is of neural origin.

According to still further features in the described preferred embodiments, the cancer is a pediatric cancer.

According to still further features in the described preferred embodiments, the cancer is a brain tumor.

According to still further features in the described preferred embodiments, the cancer is a medulloblastoma, a neuroblastoma or a pheochromocytoma.

According to still further features in the described preferred embodiments, the disease is characterized by pathological cells which comprise TrkA.

According to still further features in the described preferred embodiments, the cancer is characterized by cancer cells which comprise TrkA.

According to still further features in the described preferred embodiments, the nucleic acid construct further comprises one or more transcription control sequences being capable of driving expression of the nucleic acid sequence in pathological cells associated with the disease.

The present invention successfully addresses the shortcomings of the presently known configurations by providing a novel and effective method of treating diseases associated with abnormal apoptosis via modulation of the TrkA pathway of apoptosis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1a depicts RRS interaction of pMyr-necdin with clones of PTB fused to Ras and identified in yeast two hybrid screen. Yeast co-transfected with pMyr-necdin and empty vector are unable to grow at restrictive temperature, as shown in negative control.

FIG. 1b depicts co-immunoprecipitation of HA-Karet with necdin in COS cells, and shows that necdin interacts with a novel PTB domain protein in COS cells. Karet tagged with the HA epitope was immunoprecipitated with anti-HA antibody, the precipitated proteins were separated electrophoretically, and gel blots were incubated with anti-necdin. In control experiments the HA-Karet was substituted with vector.

FIG. 2a is an amino acid sequence diagram depicting the amino acid sequence of mouse Karet (GenBank Accession No. AAR29082.1; SEQ ID NO:1). The PTB domain (amino acid residues 60-230; SEQ ID NO:2) is underlined, and the sequence of the apoptosis-inducing PTB-domain-containing, truncation mutant of mouse Karet corresponding to amino acid residues 1-228 [SEQ ID NO:7; Karet(1-228)] is indicated in uppercase letters.

FIG. 2b depicts an amino acid sequence alignment diagram of Karet protein sequences from human [Human variant 1 [SEQ ID NO:13; GenBank Accession No. NP_{—}001025006.1; cerebral cavernous malformation 2 isoform 1; 465 amino acids long; encoded by GenBank Accession No. NM_{—}001029835 (SEQ ID NO:18)]; Human variant 2 [SEQ ID NO:3; GenBank Accession No. NP_{—}113631; cerebral cavernous malformation 2 isoform 2; 444 amino acids long; encoded by GenBank Accession No. NM_{—}031443 (SEQ ID NO:17)], and Human-long [SEQ ID NO:4; GenBank Accession No. AAQ15228; 460 amino acids long; encoded by GenBank Accession No. AF370392 (SEQ ID NO: 19)], mouse Karet (SEQ ID NO: 1; GenBank Accession No. AAR29082.1; 444 amino acids long) and rat Karet (SEQ ID NO:12; GenBank Accession No. XP_{—}001062898; 510 amino acids long) using Clustal X program.

FIG. 2c is a schematic diagram depicting the highly conserved Karet protein. The PTB domain is indicated in pink, intron positions are indicated with vertical lines showing the intron phase and exact position of the amino-acid.

FIG. 2d depicts a Northern blot analysis of Karet transcript expression in mouse tissues.

FIGS. 3a-b are Western blot analyses depicting production of a polyclonal anti-PTB antibody and expression profile of Karet. FIG. 3a depicts Hek293 cell lysates transfected with HA-Karet or GFP analyzed by Western blot with anti-HA antibody (Roche) followed with the anti-PTB serum and anti-actin antibody. FIG. 3b depicts endogenous expression of Karet examined in different cell lines as indicated. The cells were lysed in NP-40 lysis buffer and processed for SDS-PAGE analysis. The blot was first incubated with anti-PTB antibody, and then reprobed with anti-actin. Actin levels served as loading control.

FIG. 3c is a co-immunoprecipitation assay depicting co-immunoprecipitation of HA-Karet with TrkA in COS cells. Karet tagged with the HA epitope was immunoprecipitated with anti-HA antibody and the blot was incubated with anti-TrkA (RTA). In control experiments the HA-Karet was substituted with vector.

FIG. 3d is a co-immunoprecipitation assay depicting interaction of Karet and TrkA in NNR5 cells transfected with TrkA.

FIG. 3e is a co-immunoprecipitation assay depicting co-immunoprecipitation of HA-Karet with TricA in co-transfected NNR5 cells immunoprecipitated with anti-HA (Roche) and Western blot with the anti-TrkA antibody C14 (Santa Cruz).

FIG. 3f is a co-immunoprecipitation assay depicting interaction of Karet with Tyr490Ala and Tyr785Ala substitution mutants in NNR5 cells.

FIG. 3g is a co-immunoprecipitation assay depicting co-immunoprecipitation of HA-Karet with EGFR in co-transfected HEK cells. Cells were co-transfected with Ha Karet or control vector and EGFR. Cells were lysed with NP40, the HA-beads (i.e., beads conjugated to HA antibody) were blocked with Hek lysate, and elution was done for 30 minutes at 37° C. The membranes were probed with anti FLAG antibody. Note the co-immunoprecipitation of Karet with EGFR in HEK cells.

FIGS. 4a-c are co-immunoprecipitation assay results depicting the influence of NGF treatment on TrkA-Karet interaction. FIG. 4a depicts that TrkA-Karet interaction occurred prior to NGF treatment and was enhanced after ligand stimulation for 10 minutes (10 m) or 2 hours (2 h). HA-Karet was electroporated into cell line PC12, and the cells were treated with 50 ng/ml NGF for the indicated time periods in order to activate p75 and TrkA. Karet tagged with the HA epitope was immunoprecipitated with anti-HA antibody, the precipitated proteins were separated electrophoretically, and gel blots were incubated with anti-TrkA antibody (RTA). FIG. 4b depicts that necdin seems to compete with TrkA for binding to Karet. myc-Karet was electroporated into necdin stable cell line, and the cells were treated with 50 ng/ml NGF for the indicated time periods. Karet tagged with the myc epitope was immunoprecipitated with anti-myc antibody, the precipitated proteins were separated electrophoretically, and gel blots were incubated with anti-TrkA antibody (RTA). FIG. 4c depicts that the interaction between necdin and Karet, unlike that between TrkA and necdin, is robust and constant during treatment with 50 ng/ml NGF for the indicated time periods. A blot from the same experiment was stripped and re-incubated with anti-necdin antibody. The arrow indicates the glycosylated form of TrkA (molecular weight of 140 kDa).

FIG. 5a is a bar-graph of FACS data depicting 50 percent death of PC12 cells overexpressing Karet. Shown is a FACS analysis of PC12 viability 15 hours after transfection with Ha-Karet or control vector using Live/dead kit (Molecular probes), T-test=0.003.

FIG. 5b is a series of fluorescence photomicrographs depicting death of PC12 cells caused by Karet overexpression. Shown is a live/dead kit analysis of PC12 cells overexpressing Ha-Karet (left panels) versus control vector-transfected cells (right panels). Live cell are stained in green, dead cells are stained in red. Upper panels each show red fluorescence only (left picture) and green fluorescence only (right picture) pairs, and lower panels each show combined red and green fluorescence (left picture) and light microscopy images (right picture) pairs.

FIG. 5c is a pair of fluorescent histology photomicrographs depicting presence of dead cells (red fluorescence) in cultures of PC12 cells overexpressing Karet, but not in control vector-transfected cells which only show live cells (green fluorescence).

FIG. 5d is a pair of Hoechst stain histology photomicrographs depicting condensation of chromatin indicating apoptosis in PC12 cells expressing Karet, but not in control cells transfected with vector only.

FIG. 5e is a histogram depicting that Karet expression induces cell death of TrkA expressing cells, which is increased by NGF treatment and is decreased by necdin. PC12 or PC12 necdin stable cell line were electroporated with Karet or GFP control and incubated with or without 50 ng/ml NGF for 12 hours. Cells were counted and result presented as percent of control.

FIG. 5f is a viable cell count histogram depicting that the pro-apoptotic effect of Karet is TrkA dependent. NNR5 cells were electroporated with GFP with or without TrkA construct, or Karet with or without TrkA. Cells were incubated for 12 hours after electroporation and results are presented as percent of control (NNR5 transfected with GFP only, mean±SD, n=4, * p<0.05).

FIG. 6 is a cell-survival histogram depicting that Karet induces cell death through the mitochondrial pathway. PC12 cells were co-electroporated with Karet and p35, BclXI or JIP-1 and were incubated for 12 hours. PC12 electroporated with GFP instead of Karet served as control. When SB203580 and SP600125 inhibitors were used, PC12 were electroporated with Karet or GFP and incubated with 10 micromolar SB203580 or 50 nanomolar SP600125. As control for toxicity, PC12 were electroporated with Karet or GFP and incubated with DMSO (mean±SD, n=4, * p<0.05, ** p<0.01).

FIG. 7a is a bar graph depicting the viability of PC12 cells following transfection with of various Karet constructs, demonstrating that combination of Karet PTB domain with the non PTB expressed as two constructs restored some of the death effect caused by Karet. PC12 cells were transfected with Ha Karet, PTB, non PTB and their combinations. Cells were analyzed by FACS 18 hours after transfection using the dead/live kit. N=3; * p<0.05; **p<0.001;

FIG. 7b is an immunoprecipitation assay depicting that the PTB domain-containing Karet(1-228) mutant, but not Karet(228-453) co-immunoprecipitates with TrkA in NNR5 cells. Truncation mutants tagged with the HA epitope were immunoprecipitated with anti-HA antibody, the precipitated proteins were separated electrophoretically, and gel blots were incubated with anti-TrkA (RTA). The results demonstrate that the PTB domain-containing deletion mutant of Karet corresponding to amino acid residues 1-228 [SEQ ID NO: 7; Karet(1-228)], but not a deletion mutant of Karet corresponding to amino acid residues 228-453 [SEQ ID NO: 8; Karet(228-453)] interacts with TrkA in mammalian cells.

FIG. 7c is a co-immunoprecipitation assay depicting that Karet specifically interacts with a 21 amino acid residue juxtamembrane region of TrkA at position 464-484 having the sequence TLGGSSLSPTEGKGSGLQGHI (SEQ ID NO: 9). HA-Karet was co-immunoprecipitated with the indicated deletion mutants of TrkA in NNR5 cells. Karet tagged with the HA epitope was immunoprecipitated with anti-HA antibody, the precipitated proteins were separated electrophoretically, and gel blots were incubated with anti-TrkA (RTA) (IP:anti-Karet, Blot:TrkA). Cell lysates of transfected cells were subjected to Western blot analysis using the anti-TrkA antibody Lysates). As a control, the anti HA immunoprecipitates were subjected to Western blot analysis using the anti-HA antibody (IP: anti-Karet, Blot:Ha).

FIG. 8a is a co-immunoprecipitation analysis depicting interaction of Karet PTB domain with a juxtamembrane region of TrkA. Biotinylated synthetic peptide corresponding to the TrkA sequence in the juxtamembrane and control peptide were immobilized on streptavidin beads and then used to precipitate recombinant Karet protein. Precipitates were washed, eluted and bound Karet was visualized by Western blot with anti-His antibody.

FIG. 8b is an in-vitro affinity co-precipitation Western blot depicting that the isolated N-terminal PTB domain of Karet specifically interacts with a 21 amino acid residue juxtamembrane region of TrkA having the sequence Biotinylated synthetic peptides corresponding to a 27 amino acid residue juxtamembrane region of TrkA having the sequence MSLHFMTLGGSSLSPTEGKGSGLQGHI (SEQ ID NO: 10), and two control peptides were immobilized on streptavidin beads, and then used to precipitate recombinant PTB domain-containing Karet(1-228) truncation mutant. Precipitates were washed, eluted and bound Karet(1-228) was visualized by Western blot with Karet antibody. Note that Karet(1-228) is precipitated by the TrkA peptide, but not by two control peptides. The rightmost lane shows Karet(1-228) run as a standard on the gel.

FIG. 9 shows that the cell death effect of Karet is dependent on the protein expression levels in cells. The lower panel is a histogram depicting that Karet-mediated cell death correlates well with expression levels of the Karet protein. PC12 Tet-on cells were electroporated with Ha-Karet cloned under inducible promoter and incubated for 24 hours with different concentrations of doxycycline. Cells transfected with Karet but not treated with doxycycline served as control. Cells were counted and the results were determined as percent of control. The upper panel is a control Western blot depicting increased Karet expression with increased concentrations of doxycycline. Cells transfected with Karet and treated with doxycycline were lysed in NP-40 buffer and subjected to SDS-PAGE analysis. Similar amount of protein was loaded and blotted for Karet expression with Ha antibody. Proteins levels were checked with anti-tubulin antibody.

FIGS. 10a-b show that expression of Karet enhances the phosphorylation state of TrkA. FIG. 10a is a bar-graph depicting that inhibition of TrkA phosphorylation prevents Karet-mediated cell death. PC 2 cell line were electroporated with Karet or GFP and incubated for 12 hours with different concentration of the TrkA phosphorylation inhibitor K252A. Cells were counted and data analyzed as percent of survival Karet expressing cells versus GFP expressing cells. FIG. 10b is a set of immunoprecipitation assay results showing that NGF stimulation of Karet-expressing cells induces TrkA phosphorylation. PC12 6.24 cells were electroporated with Karet or GFP. The cells were treated with 50 ng/ml NGF for different time periods. TrkA was immunoprecipitated from the cells, the precipitated proteins were separated electrophoretically, and gel blots were incubated with anti-phospho-Tyr antibody. The general level of TrkA was detected with RTA antibody.

FIGS. 11a-b show production and purification of anti-Karet antibody. FIG. 11a is a Western blot analysis depicting detection of affinity purified Karet-GST with anti-Karet antibody. Karet protein was expressed as fusion to GST tag and purified on a GST column. 8 mg of purified GST-Karet was coupled through amino-groups to NHS-activated HP column. Serum was purified and antibody elution peak was combined and frozen with glycerol. 500 ng of purified GST-Karet was processed by SDS-PAGE and incubated with 20 ng/ml of Karet antibody. FIG. 11b is an immunoprecipitation analysis depicting detection of HA-Karet with anti-Karet antibody. COS cells transfected with HA-Karet or GFP were incubated with anti-Ha antibody and immunoprecipitates were blotted with anti-Karet antibody. The blot was reprobed with anti-HA antibody. A similar protein species was detected in both cases.

FIG. 12 is a pair of Western immunoblotting analyses showing that various neuroblastoma and medulloblastoma cell lines endogenously express Karet. Different cell lines including COS, NNR5 and PC12 were lysed in NP-40 lysis buffer and processed for SDS-PAGE analysis. Blots of the separated proteins were first incubated with anti-Karet antibody, then reprobed for detection of TrkA expression. Beta-tubulin level analysis served as loading control.

FIG. 13 is a set of immunoprecipitation assays depicting that Karet co-immunoprecipitates with TrkA from TrkA overexpressing medulloblastoma cells. Cells treated with or without NGF for 57 hours were lysed in NP40 buffer. TrkA was immunoprecipitated overnight with pan-Trk antibody, incubated with protein-G beads for 1 hour and the precipitated protein was further subjected to immunoblotting analysis with antibody against Karet. Medulloblastoma cell lysates not transfected to express TrkA were incubated with beads as control for non-specific binding of Karet.

FIGS. 14a-b are Western immunoblotting assays depicting expression levels of Karet and TrkA in human neuroblastoma tumors. Samples were separated by SDS-PAGE and blotted with antibodies against Karet and TrkA, and against beta-tubulin as a normalization control. A minimum of 100 micrograms of protein was loaded into each lane.

FIG. 15 is immunohistology photomicrograph depicting a poorly differentiated neuroblastoma, with all cells Karet-negative (Magnification ×400). (Magnification ×200).

FIG. 16 is immunohistology photomicrograph depicting an intermixed ganglioneuroblastoma. Note prominent Karet-positive staining of the large differentiating cells (Magnification ×200).

FIG. 17 schematically depicts the constructs of the PTB domain only polypeptide, PTB domain having the F217V missense mutation (substitution of phenyl alanine to valine at amino acid 217 in the full-length mouse Karet protein of SEQ ID NO:1 [corresponding F217V in SEQ ID NO:3 (human variant 2) and 4 (human long), and F238V in SEQ ID NO:13 (human variant 1)], and the Karet polypeptide having the F217V missense mutation. The PTB domain is demonstrated as an orange box.

FIGS. 18a-b are FACS (FIG. 18a) and Western blot (FIG. 18b) analyses demonstrating that Karet mutants do not cause death in PC12 cells. FIG. 18a—PC12 were transfected with Ha Karet, Karet F217V, PTB F217V or a control vector (pcDNA3) and 18 hours after transfection the effect of Karet or Karet mutants on cell death was determined using the dead/live kit and FACS analysis. Results are presented in percentages as compared to cells transfected with the control vector. N=3 **P<0.001. Note the significant reduction in the viability of cells transfected with the wild-type Karet coding sequence but not in Karet mutants (Karet F217V or PTB F217V). FIG. 18b—Cells were lysed 24 hours after transfection, fractionated on SDS-PAGE and immunoblot for anti Ha (Covance). Note the presence of Karet proteins (immuno-reactive with anti-Ha 11 antibody) in cells transfected with the Ha-Karet, Ha PTB F217V and Ha Karet F217V, but not in cells transfected with the empty vector.

FIG. 19 is a histogram depicting FACS analysis of Karet-induced death. PC12 cells were co-transfected with Karet and the PTB, non-PTB or the F217V mutant at different ratios (1:1, 1:2, 1:3) and 18 hours after transfection the cells were analyzed by FACS using the dead/live kit. Note that co transfection of the non-PTB or Karet F217V along with Karet reduced Karet-induced death.

FIGS. 20a-q are immunochemistry (FIGS. 20a-p) and FACS (FIG. 20q) analyses of myristylated constructs of Karet, PTB and the non-PTB domain in PC12 (FIGS. 20a-p) or NNR5 (FIG. 20q) cells. Myristylated constructs of Karet (FIGS. 20e-h), PTB (FIGS. 20i-l) and the non-PTB domain (FIGS. 20m-p) or non-myristylated construct of Karet (FIGS. 20a-d) were transfected to PC12 cells and immunochemistry of the expressed constructs was performed using HA-antibody (FIG. 20a), the PTB-antibody (FIGS. 20e, i and m) or Lamin (a nuclear membrane protein)-antibody (FIGS. 20b, f, j, and n). Merged images of the two-color immunocytochemistry are shown in FIG. 20d (for anti-Ha and anti-lamin antibodies) or FIGS. 20h l, and p (for anti-PTB and anti-lamin antibodies). Phase contrast images of the cells are shown in FIGS. 20c, g, k and o. Note the cytoplasmic expression of non-myristylated Karet (FIG. 20a) as compared to the nuclear membrane expression of lamin (FIG. 20b). Also note the membranal expression of myristylated karet (FIG. 20e), PTB (FIG. 20i) or non-PTB domain (FIG. 20m). FIG. 20q—FACS analysis of the transfected NNR5 cells. NNR5 cells were transfected with pcDNA3, Ha Karet and TrkA, Myr-Karet, Myr-PTB or Myr-non PTB constructs and the effect on cell viability was determined using calcein (the dead/Live kit). Note the significant reduction of cell viability in NNR5 cells transfected with Karet and TrkA as compared to the myristylated constructs which were transfected alone (without TrkA). * p<0.05 **p<0.001.

FIGS. 21a-q are photomicrographs of Hek cells 24 hours after transfection with karet, Trk receptors, EGFR and/or P75 constructs. Hek cells were transfected with Karet or control vector and Trk receptors (TrkA, TrkB or TrkC), EGFR, p75 and 24 hours after transfection the cells were visualized by microscope at 10× magnitude for transfection-induced change in morphology, demonstrating transfection related cell death. FIG. 21a—cells transfected with TrkA and pcDNA3; FIG. 21b—TrkA and Karet; FIG. 21c—TrkA and Karet F217V; FIG. 21d—Karet and pcDNA4; FIG. 21e—Karet F217V and pcDNA4; FIG. 21f—TrIcA and pcDNA3; FIG. 21g—TrkB and pcDNA3; FIG. 21h—TrkC and pcDNA3; FIG. 21i—TrkA and Karet;

FIG. 21j—TrkB and Karet; FIG. 21k—TrkC and Karet; FIG. 21l—Karet and pcDNA3; FIG. 21m—TrkC (low concentration) and pcDNA3; FIG. 21n—EGFR-Flag and pcDNA3; FIG. 21o—p75 and pcDNA3; FIG. 21p—EGFR-Flag and Ha-Karet; FIG. 21q—p75 and Ha-Karet. Note the effect of transfection of Karet and TrkA (FIGS. 21b and i), Karet F217V and TrkA (FIG. 21c) and Karet and EGFR (FIG. 21p) on cell morphology, demonstrating transfection-induced cell death.

FIGS. 22a-b are histograms depicting the effect of co-transfection of Karet and TrkA on cell death in Hek (FIG. 22a) or NNR5 (FIG. 22b) cells. FIG. 22a—Hek cells were co-transfected with Karet or control vector and TrkA, TrkB and TrkC. Viability of the cells was measured 24 hours after transfection by XTT. *p<0.05; FIG. 22b—NNR5 cells were co-transfected with Karet or control vector and TrkA, TrkB and TrkC. Cells were analyzed by FACS 24 hours after transfection using the dead/live kit. ** p<0.001.

FIG. 23 is a histogram depicting the effect of co-transfection of EGFR and Karet on cell death in Hek cells. Hek cells were co-transfected with Karet or control vector and p75, EGFR and PTB. Viability of the cells was measured 24 hours after transfection by XTT. ** p<0.001.

FIGS. 24a-d depict co IP of Ha Karet with TrkA, TrkB, TrkC, p75 from co-transfected Hek cells. Cells were co-transfected with Ha Karet or control vector and TrkA, TrkB, TrkC or p75. Cells were lysed with NP40, the HA-beads (beads conjugated to anti-HA antibody) were blocked with Hek lysate, and elution was done for 30 minutes at 37° C. Proteins from anti-HA-IP (FIGS. 24a and b) or from cell lysates without IP (FIGS. 24c and d) were subjected to Western blot analysis and the membranes were probed with anti pan trk (FIGS. 24a and c) or anti-p75 (FIGS. 24b and d) antibodies. Note the specific co-IP of Karet with TrkA (FIG. 24a, lane 1), Karet with TrkB (FIG. 24a, lane 3), Karet with TrkC (FIG. 24a, lane 5) and the lack of IP between karet and p75 (FIG. 24b, lane 7).

FIGS. 25a-b are photomicrographs depicting RRS screen using the ADH-non PTB as a bait with cDNA library of embryonic mouse brain. FIG. 25a—Positive selection—the colonies were grown on selective medium (galactose-LUT) at a selective temperature (36° C.). Left-Fatso, right-control candidate. FIG. 25b—Negative selection—the colonies were grown on glucose medium at a selective temperature (36° C.). Left-Fatso, right-control candidate (pulse positive).

FIGS. 26a-e depict co IP of Ha Karet, Karet F217V and PTB and with TrkA from co-transfected Hek cells. Cells were co-transfected with TrkA and Ha Karet (lane 1, Karet), Ha Karet F217V (lane 2, F217V), Ha PTB (lanes 3 and 4, PTB) or control vector (pcDNA3) (lane 5), or remained un-transfected (lane 6, Ab alone). 24 hours after transfection the cells were lysed with NP40, the HA-beads (beads conjugated to anti HA antibody) were blocked with Hek lysate, and elution was done for 30 minutes in 37° C. FIGS. 26a, b and c are Western blot analyses of anti-HA IP using anti Trk antibody (pan Trk; FIG. 26a) or anti HA antibody (WB HA; FIG. 26b—short exposure, FIG. 26c—long exposure). FIGS. 26d and e are Western blot analyses of the cell lysates using anti Trk antibody (pan Trk; FIG. 26d) or anti HA antibody (WB HA; FIG. 26e). Note the specific co-immunoprecipitation of Karet, the F217V Karet mutant, or the PTB domain alone with TrkA, demonstrating that the PTB domain is capable of binding to TrkA and that the missense mutation (F217V) does not interfere with binding of Karet with TrkA.

FIG. 27 is a schematic illustration of a potential model by which Karet (composed of the PTB and non-PTB domains) mediates cell death. NGF binds to the TrkA receptor on the cell membrane, which interacts with Karet via the PTB domain, and transduces a signal for cell death via Factor “X”. Without being bound by any theory, Fatso, which was identified as a co-interacting with the non-PTB domain of karet (see Table 3, Example 2 of the Examples section which follows) can function as factor “X”.

### DESCRIPTION OF THE PREFERRED EMBODIMENTS

The present invention is of a method of treating a disease associated with abnormal apoptosis in a subject. Specifically, the present invention can be used to induce apoptosis of a pathological cell population of a subject. As such, the present invention can be used to treat any disease, such as a cancer, which is amenable to treatment via killing of a pathological cell population of a subject. In particular, the present invention can be used to treat a cancer, in particular a cancer of neural origin.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Novel and optimal treatments are urgently required for treatment of cancers of neural origin, in particular pediatric tumors such as medulloblastoma or neuroblastoma. In view of the art-recognized role of trk proteins in the regulation of trk-dependent cell death in such tumors, a potentially optimal strategy for treating such diseases would be to identify molecules involved in regulation of trk-dependent cell death, and to suitably regulate their levels/activity so as to induce therapeutic cellular apoptosis.

Various approaches for treating pediatric tumors of neural origin via induction of trk-mediated cell death have been described by the prior art. One approach involves retrovirally transducing medulloblastoma tumor cells to express TrkA, optionally in combination with administration of nerve growth factor (NGF; Muragaki Y. et al., 1997). Another approach involves transfecting glioma tumor cells to express trkA, optionally in combination with administration of NGF (Kokunai T. et al., 1999. J. Neurooncol. 42:23-34). Yet another approach involves delivery of the Trk inhibitor CEP-751 to neuroblastoma or medulloblastoma tumor cells, optionally in conjunction with transfection of tumor cells to express TrkB (Evans A E. et al., 1999. Clin Cancer Res. 5:3594-602). Still another approach proposes treating medulloblastoma using regulation of putative novel Ras and/or Raf signaling pathways (Chou et al., 2000).

However, all such prior art approaches suffer from various drawbacks, including never having been attempted in a subject having an endogenous tumor, nor in any human patient.

Thus, all prior art approaches involving modulation of trk mediated cell death have failed to provide a proven method of treating neural malignancies, such as pediatric tumors of neural origin.

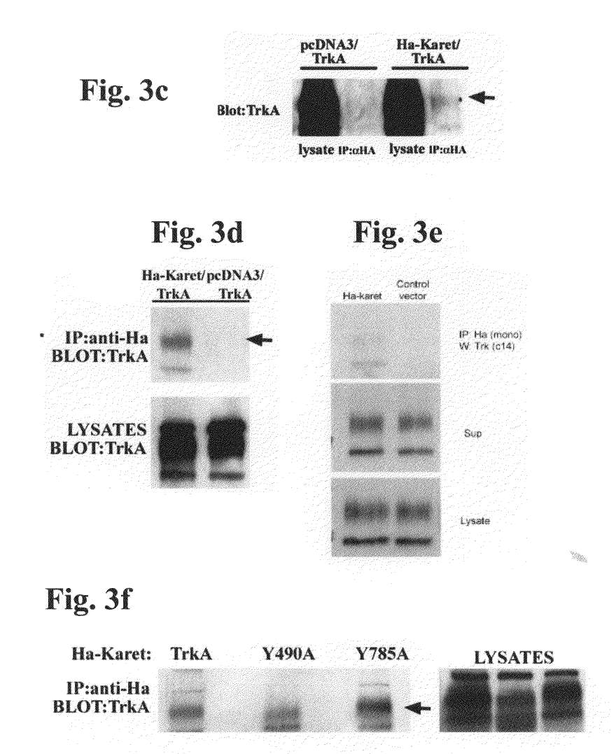

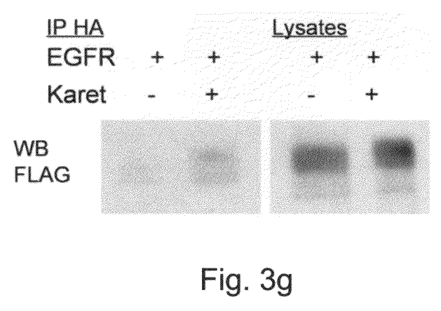

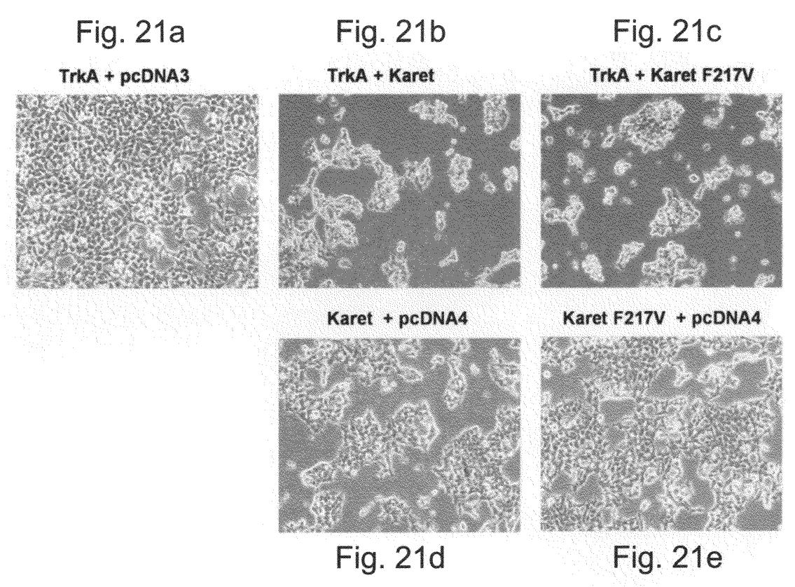

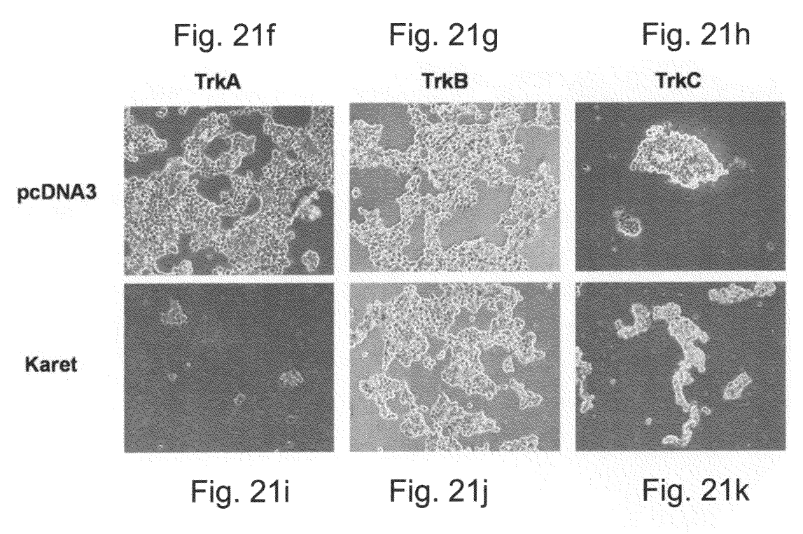

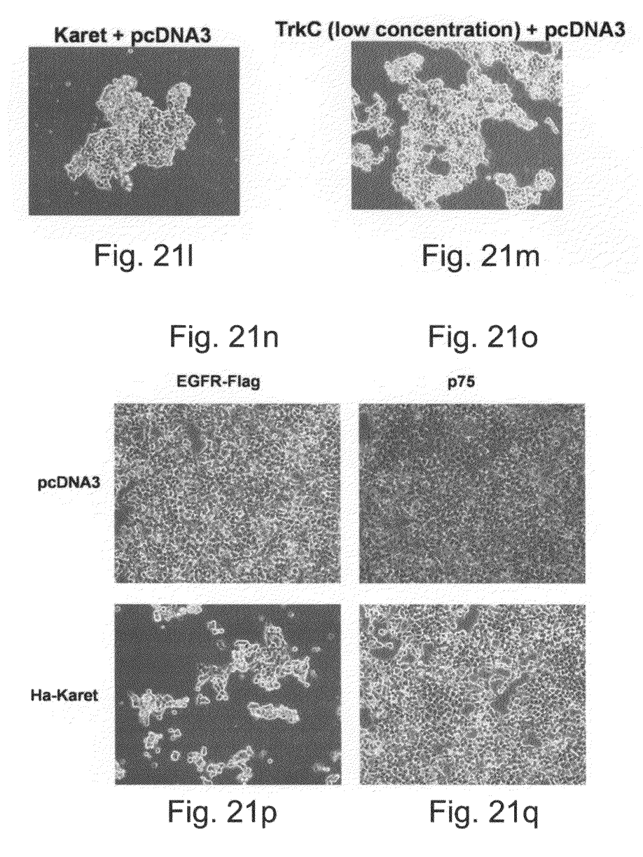

While reducing the present invention to practice, as described in Example 1 of the Examples section below, it was uncovered that the protein Karet [which is also known as OSM (osmosensing scaffold for MEKK3) in mouse or CCM2 (cerebral cavernous malformation 2) in human] interacts with TrkA in tumor cells of neural origin, in particular via an interaction between presently identified active portions of these proteins, and that expression of Karet in tumor cells of neural origin which express TrkA induces the death of such cells. In addition, as is further shown in FIGS. 3g and 21a-q and described in Examples 1 and 8 of the Examples section which follows, the protein Karet (CCM2) is also capable of binding to EGFR and inducing cell death.

As such, the present invention provides a novel and effective method of inducing death of cells, such as tumor cells of neural origin.

Thus, the method of the present invention can be used to treat a cancer, such as a cancer of neural origin.

Thus, according to the present invention there is provided a method of treating a disease which is associated with abnormal apoptosis in a subject in need thereof. The method is effected by administering to the subject a therapeutically effective amount of at least one agent which is capable of modulating an interaction between at least an active portion of TrkA or EGFR and at least an active portion of CCM2 (Karet).

The term “treating” refers to inhibiting, preventing, curing, reversing, attenuating, alleviating, minimizing, suppressing or halting the deleterious effects of a disease and/or causing the reduction, remission, or regression of a disease. Those of skill in the art will understand that various methodologies and assays can be used to assess the development of a disease, and similarly, various methodologies and assays may be used to assess the reduction, remission or regression of the disease.

According to one preferred embodiment of the method of the present invention, the agent is selected capable of increasing and/or stabilizing the interaction between an active portion of TrkA or EGFR of the present invention and an active portion of CCM2 (Karet) of the present invention.

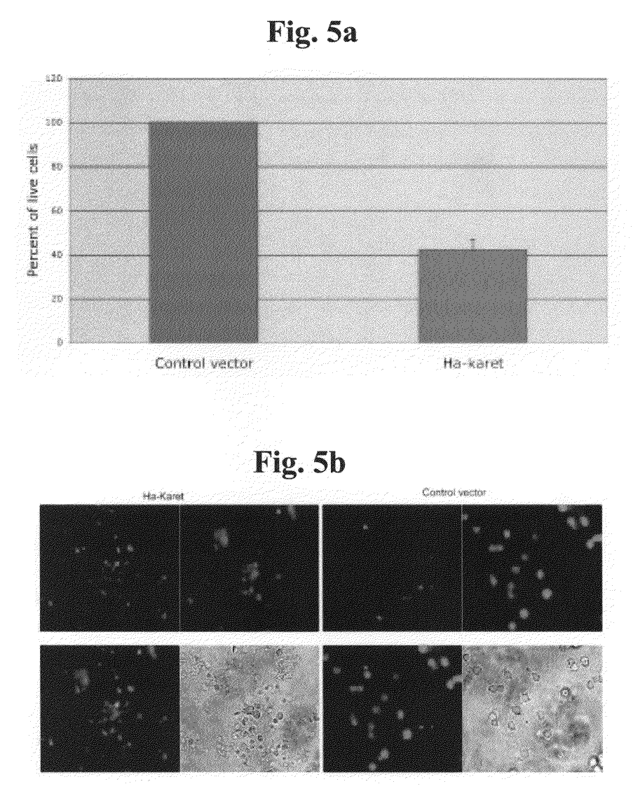

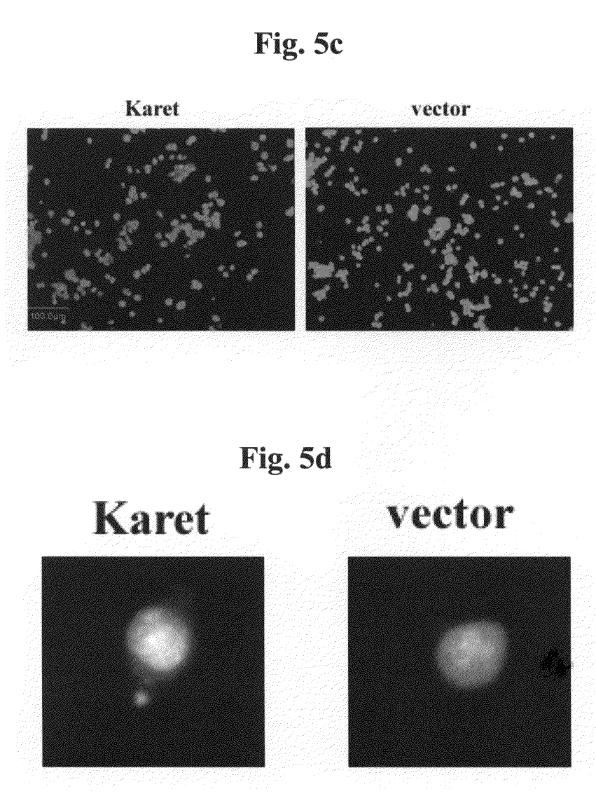

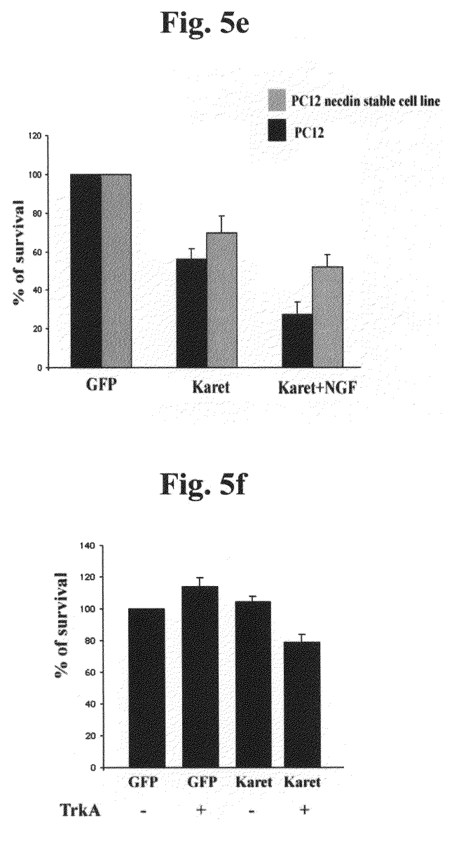

Without being bound to a paradigm, the present inventors are of the opinion that the experimental results presently disclosed in Example 1 of the Examples section below reveal that upregulating an interaction between an active CCM2 (Karet) portion of the present invention and TrkA in a pathological cell of the present invention can function to induce apoptosis of the pathological cell via the art-recognized TrkA-mediated biochemical pathway of apoptosis induction. Namely, Karet, via an active Karet portion of the present invention, and TrkA have the capacity to specifically associate with each other (for example, refer to FIGS. 3c-g and 7b) in pathological cells which are associated with abnormally decreased apoptosis, such as cancer cells, to thereby kill such pathological cells (for example, refer to FIGS. 5a-f and 7a).

The subject according to the present invention is preferably a mammal and most preferably a human.

As used herein the term “CCM2” refers to at least an active portion of the CCM2 polypeptides [e.g., the protein product of the gene MGC4607 (Mammalian Gene Collection # 4607; Strausberg et al., 2002), and has been termed in the art “cerebral cavernous malformation 2” (CCM2) in humans (Denier et al., 2004; Liquori et al., 2003), and “osmosensing scaffold for MEKK3” (OSM) in the mouse; Uhlik et al., 2003)] which is sufficient to induce cell death. Examples of CCM2 polypeptides are provided in SEQ ID NOs:1, 3, 4, 12 or 13.

As used herein the term “TrkA” refers to at least an active portion of the TrkA polypeptide (e.g., GenBank Accession Nos. P04629, NP_{—}002520, NP_{—}001007205, NP_{—}067600) which is sufficient to induce cell death. Preferably, the active portion of TrkA is a TrkA amino acid sequence which comprises SEQ ID NO:9 or 10. TrkA is a well known protein in the art which is involved in apoptosis signaling in cells such as tumor cells of neural origin (refer, for example, to Bono et al., 1997; Chou et al., 2000; Lucarelli et al., 1997; Muragaki et al., 1997; Rubin and Segal, 2003; Woo et al., 2004; Yan et al., 2002).

Thus, preferably, depending on the application and purpose, as described hereinbelow, administering to the subject a therapeutically effective amount of at least one agent which is capable of modulating an interaction between at least an active portion of TrkA and at least an active portion of Karet is effected by administering to the subject an active Karet portion of the present invention, and/or administering to the subject TrkA.

Any of various active Karet portions may be employed to effectively practice embodiments of the present invention.

According to the teachings of the present invention, the active portion of Karet is preferably capable of interacting with TrkA and/or EGFR in a pathological cell expressing these transmembrane proteins and associated with the disease, and of inducing death of the pathological cell.

The active Karet portion is preferably a polypeptide having an amino acid sequence which has at least 70 percent homology to the amino acid sequence set forth by SEQ ID NO: 1 or, preferably is a polypeptide having an amino acid sequence which has at least 70 percent homology to the amino acid sequence set forth by SEQ ID NO: 7. The amino acid sequence set forth by SEQ ID NO: 1 corresponds to full-length mouse Karet protein (GenBank Accession No. AY442689), and the amino acid sequence set forth by SEQ ID NO: 7 corresponds to amino acid residue coordinates 1-228 of mouse Karet protein.

As used herein, the “percent homology” of a test amino acid sequence (e.g. active portion of Karet) to a reference amino acid sequence (e.g. SEQ ID NO: 1 or 7) corresponds to the “Positives” output obtained for the test sequence when using the reference sequence as input to perform a standard/default search of the protein-protein BASIC LOCAL ALIGNMENT SEARCH TOOL (BLAST) [blastp] software of the National Center for Biotechnology Information (NCBI; www.ncbi.nlm.nih.gov/BLAST/).

Preferably, the amino acid sequence having at least 70 percent homology to the amino acid sequence set forth by SEQ ID NO: 1, has a percent homology to the amino acid sequence set forth by SEQ ID NO: 1 of 75 percent, more preferably 80 percent, more preferably 85 percent, more preferably 90 percent, more preferably 95 percent, more preferably 96 percent, more preferably 97 percent, more preferably 98 percent, more preferably 99 percent, and most preferably 100 percent.

Most preferably, the amino acid sequence having at least 70 percent homology to the amino acid sequence set forth by SEQ ID NO: 1 has the amino acid sequence set forth by SEQ ID NO: 1.

Where the subject is a human, the amino acid sequence having at least 70 percent homology to the amino acid sequence set forth by SEQ ID NO: 1 corresponds to the polypeptide whose amino acid sequence is set forth by GenBank Accession No. NP_{—}113631 (SEQ ID NO: 3), GenBank Accession No. AAQ15228 (SEQ ID NO: 4) or GenBank Accession No. NP_{—}001025006 (SEQ ID NO:13), which correspond to 444, 460 and 465 amino acid residue forms of human Karet (CCM2), respectively.

Preferably, the amino acid sequence having at least 70 percent homology to the amino acid sequence set forth by SEQ ID NO: 7, has a percent homology to the amino acid sequence set forth by SEQ ID NO: 7 of 75 percent, more preferably 80 percent, more preferably 85 percent, more preferably 90 percent, more preferably 95 percent, more preferably 96 percent, more preferably 97 percent, more preferably 98 percent, more preferably 99 percent, and most preferably 100 percent.

Where the subject is a human, the amino acid sequence having at least 70 percent homology to the amino acid sequence set forth by SEQ ID NO: 7 corresponds to the polypeptide having coordinates 1-228 of the amino acid sequence which is set forth by GenBank Accession No. NP_{—}113631 or AAQ15228, which correspond to 444 and 460 amino acid residue forms of human Karet, respectively. Alternately, where the subject is a human, the amino acid sequence having at least 70 percent homology to the amino acid sequence set forth by SEQ ID NO: 7 corresponds to the polypeptide having coordinates 1-197 of the amino acid sequence which is set forth by GenBank Accession No. NP_{—}113631 or AAQ15228.

The active Karet portion may advantageously comprise a PTB domain of Karet, preferably defined using SMART software. Examples of suitable Karet PTB domains comprise a polypeptide having an amino acid sequence set forth by SEQ ID NO: 2, i.e. corresponding to amino acid residues 60-230 of the amino acid sequence set forth by SEQ ID NO: 1. Preferably, where the subject is a human, examples of suitable PTB domains comprise a polypeptide having an amino acid sequence corresponding to amino acid residues 60-230 of the amino acid sequence of a human Karet polypeptide, preferably corresponding to GenBank Accession No. NP_{—}113631 or AAQ15228.

A suitable Karet PTB domains may comprise a polypeptide having an amino acid sequence corresponding to amino acid residues 60-228 of the amino acid sequence set forth by SEQ ID NO: 1

According to the teachings of the present invention, the active portion of TrkA is preferably a polypeptide having an amino acid sequence which has at least 70 percent homology to the amino acid sequence set forth by SEQ ID NO: 10 or, more preferably, is a polypeptide having an amino acid sequence which has at least 70 percent homology to the amino acid sequence set forth by SEQ ID NO: 9.

Preferably, the amino acid sequence having at least 70 percent homology to the amino acid sequence set forth by SEQ ID NO: 10, has a percent homology to the amino acid sequence set forth by SEQ ID NO: 10 of 75 percent, more preferably 80 percent, more preferably 85 percent, more preferably 90 percent, more preferably 95 percent, more preferably 96 percent, more preferably 97 percent, more preferably 98 percent, more preferably 99 percent, and most preferably 100 percent.

Preferably, the amino acid sequence having at least 70 percent homology to the amino acid sequence set forth by SEQ ID NO: 9, has a percent homology to the amino acid sequence set forth by SEQ ID NO: 9 of 75 percent, more preferably 80 percent, more preferably 85 percent, more preferably 90 percent, more preferably 95 percent, more preferably 96 percent, more preferably 97 percent, more preferably 98 percent, more preferably 99 percent, and most preferably 100 percent.

As described hereinabove, depending on the application and purpose, the method of the present invention may be effected by administering to the subject any combination of an active Karet portion of the present invention, and/or TrkA. According to the embodiment of the present invention exemplified in Example 1 of the Examples section below, an active Karet portion of the present invention and/or TrkA are administered to pathological cells to be killed such that the pathological cells will substantially express/comprise both the active Karet portion and TrkA, to thereby induce death of the pathological cells.

Where the pathological cells to be killed endogenously express/comprise TrkA, the embodiment the present invention exemplified by Example 1 of the Examples section below may be suitably practiced by administering to the subject an active Karet portion of the present invention. As is described and illustrated in Example 1 of the Examples section which follows (e.g. refer to FIGS. 5c and 5e), administration of an active Karet portion of the present invention, according to the teachings of the present invention, to pathological cells, such as TrkA-expressing cancer cells of neural origin results in killing of such pathological cells, as exemplified using cancerous TrkA-expressing pheochromocytoma cells.

According to the embodiment the present invention exemplified by Example 1 of the Examples section below, where the pathological cells to be killed do not substantially express/comprise TrkA nor a functional Karet molecule, the method may be suitably practiced by administering to the subject both an active Karet portion of the present invention as well as an active portion of TrkA. As is described and illustrated in Example 1 of the Examples section which follows (e.g. refer to FIG. 5f), administration of an active Karet portion of the present invention and of TrkA to pathological cells, such as cancer cells of neural origin, results in killing of such pathological cells, as exemplified in TrkA-negative pheochromocytoma cells.

In case the pathological cells to be killed do not substantially express/comprise EGFR nor a functional Karet molecule, the method may be suitably practiced by administering to the subject both an active Karet portion of the present invention as well as an active portion of EGFR (see for example, Example 8 of the Examples section which follows and FIGS. 21a-q).

As used herein the phrase “active portion of EGFR” refers to any EGFR amino acid sequence which is capable of inducing cell death.

EGFR, epidermal growth factor receptor (EGFR, located at human chromosome 7p12) is a transmembrane protein with intrinsic tyrosine kinase activity expressed primarily on cells of epithelial origin. EGFR regulates cell growth in response to activation by EGF and transforming growth factor- (TGF-) binding. EGFR isoforms which can be used along with the present invention include isoform a [GenBank Accession No. NP_{—}005219.2 (SEQ ID NO:22) which is encoded by GenBank Accession No. NM_{—}005228.3 (SEQ ID NO:23)], isoform b [GenBank Accession No. NP_{—}958439.1 (SEQ ID NO:24) which is encoded by GenBank Accession No. NM_{—}201282.1 (SEQ ID NO:25)], isoform c [GenBank Accession No. NP_{—}958440.1 (SEQ ID NO:26) which is encoded by GenBank Accession No. NM_{—}201283.1 (SEQ ID NO:27)] and isoform d [GenBank Accession No. NP_{—}958441.1 (SEQ ID NO:28) which is encoded by GenBank Accession No. NM_{—}201284.1 (SEQ ID NO:29)].

The phrase “cell death” as used herein refers to killing of cells as a result of necrosis (a result of acute tissue injury and/or inflammatory response) and/or apoptosis (programmed cell death). Preferably, “cell death” according to the present invention is effected via apoptosis.

Methods of qualifying active portions can employ cell death assay, where a cancerous cell expressing EGFR/TrkA is treated with peptide (or nucleic acid sequences encoding the peptides) and cell death is induced with or without EGF or NGF. Cell death may be determined by methods well known in the art such as caspase assays, the Ethidium homodimer-1 staining (Invitrogen-Molecular Probes), the Tunnel assay (Roche, Basel, Switzerland), the Live/dead viability/cytotoxicity two-color fluorescence assay (Molecular Probes, Inc., L-3224, Eugene, Oreg., USA), FACS analysis [using molecules capable of specifically binding cells undergoing apoptosis, such as propidium iodide and Annexin V], and those of skills in the art are capable of assessing such levels in order to determine the standards of normal levels.

Detection of Karet, TrkA and/or EGFR in pathological cells may be effected according to any of various standard art methods, for example via Western immunoblotting of cell lysates, as described in Example 1 of the Examples section which follows as well as by FACS analysis, immunohistochemistry and the like. Alternately, since the nucleotide sequences of the genes encoding Karet, TrkA and EGFR, and since the amino acid sequences of these polypeptides are known, the expression of these polypeptides in pathological cells may be inferred by detection of mRNA transcripts thereof using common art methods such as RTo-PCR, RNA-in situ hybridization, Northern blot analysis and in situ RT-PCR.

As is shown in Table 3 and described in Example 2 of the Examples section which follows, using the non-PTB domain as a bait with an embryonic mouse brain library Fatso was identified as a Karet interactor.

Fatso [GenBank Accession No. NM_{—}001080432 (SEQ ID NO:20) for nucleic acids and GenBank Accession No. NP_{—}001073901.1 (SEQ ID NO:21) for amino acids] is a candidate gene involved in processes of programmed cell death, craniofacial development, and establishment of left-right asymmetry. To date, Fatso was not known to interact with Karet and to be involved in Karet-induced cell death and as such use of same for treating cancer particularly of EGFR/TrkA induced cancers (e.g., brain tumors).

While further reducing the present invention to practice, the present inventors have uncovered a method of treating cancer (e.g. brain tumor) using Fatso.

Thus, according to another aspect of the present invention there is provided a method of treating a cancer in a subject in need thereof. The method is effected by administering to the subject a therapeutically effective amount of Fatso in cells of the cancer of the subject, thereby treating the cancer in the subject.

Preferably, the Fatso comprises a polypeptide having an amino acid sequence as set forth by SEQ ID NO:16 or 21.

The polypeptides of present invention can be biochemically synthesized such as by using standard solid phase techniques. These methods include exclusive solid phase synthesis, partial solid phase synthesis methods, fragment condensation and classical solution synthesis. These methods are preferably used when the peptide is relatively short (i.e., 10 kDa) and/or when it cannot be produced by recombinant techniques (i.e., not encoded by a nucleic acid sequence) and therefore involve different chemistry.

Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

In cases where large amounts of the polypeptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680, Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

Recombinant polypeptides can be generated using various host cells such as bacterial cells, yeast cells, mammalian cells and the like.

Depending on the vector and host system used for production, resultant polypeptides of the present invention may either remain within the cell, secreted into the fermentation medium, secreted into a space between two cellular membranes, such as the periplasmic space in E. coli; or retained on the outer surface of a cell or viral membrane.

Following a predetermined time in culture, recovery of the recombinant polypeptide is effected. The phrase “recovery of the recombinant polypeptide” used herein refers to collecting the whole fermentation medium containing the polypeptide and need not imply additional steps of separation or purification.

Thus, polypeptides of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

The polypeptide of the present invention is preferably retrieved in “substantially pure” form. As used herein, the phrase “substantially pure” refers to a purity that allows for the effective use of the recombinant polypeptide (e.g., CCM2) in killing pathological cells (e.g., cancerous cells) and treating the disease (e.g., brain cancer).

Combinatorial chemical or peptide libraries may be used to screen a plurality of agents.

It will be appreciated that when utilized along with automated equipment, the above-described method can be used to screen multiple agents both rapidly and easily.

A therapeutic agent of the present invention may be administered to the subject in any of various ways.

Preferably, administering to the subject a therapeutic agent of the present invention is effected so as to achieve delivery of the agent to pathological cells which are associated with the disease in such a way as to achieve therapeutic killing of such cells.

According to the teachings of the present invention, administering a therapeutic agent such as a therapeutic polypeptide or therapeutic nucleic acid of the present invention to the subject is preferably effected by administering to the subject a nucleic acid construct comprising a nucleic acid sequence which suitably encodes the therapeutic agent (by gene therapy means). Preferably, the construct further comprises one or more transcription control sequences which are capable of driving expression of the nucleic acid sequence encoding the therapeutic agent (e.g., CCM2, TrkA, NGF and/or EGFR) in pathological cells targeted for killing.

It will be well within the purview of the ordinarily skilled artisan to adapt the teachings of the present invention in accordance with routine art methodology so as to prepare a suitable nucleic acid construct of the present invention, administer the construct to the subject in such a way as to achieve delivery of the construct to pathological cells to be killed in a subject of the present invention, and to achieve consequent expression therein of a therapeutic agent of the present invention, to thereby kill pathological cells associated with the disease in the subject.

For example, it will be well within the purview of the ordinarily skilled artisan to produce suitable nucleic acid constructs for expression of TrkA, and to use such constructs to genetically modify cells, such as pathological cells of the present invention, to express TrkA, such as human TrkA, as clearly exemplified in Example 1 of the Examples section which follows and in the art (refer, for example, to Katzir I. et al., 2002. A quantitative bioassay for nerve growth factor, using PC12 clones expressing different levels of trkA receptors. J Mol. Neurosci. 18:251-64; Koo P H, Qiu W S., 1994. Monoamine-activated alpha 2-macroglobulin binds trk receptor and inhibits nerve growth factor-stimulated trk phosphorylation and signal transduction. J Biol Chem. 269:5369-76).

Ample guidance for suitably performing gene therapy according to the teachings of the present invention, via delivery of a nucleic acid construct, such as a nucleic acid construct of the present invention, to tumor cells, such as brain tumor cells, for delivery of desired therapeutic agents of the present invention, is provided in the literature of the art (refer, for example, to: Yoshida J, Mizuno M., 2003. Clinical gene therapy for brain tumors. Liposomal delivery of anticancer molecule to glioma. J Neurooncol. 65:261-7; Kew Y, Levin V A., 2003. Advances in gene therapy and immunotherapy for brain tumors. Curr Opin Neurol. 16:665-70; Rainov N G, Ren H., 2003. Gene therapy for human malignant brain tumors. Cancer J. 9:180-8; Rainov N G, Kramm C M., 2001. Vector delivery methods and targeting strategies for gene therapy of brain tumors. Curr Gene Ther. 1:367-83; Palu G. et al., 2001. Gene therapy of brain and endocrine tumors. Croat Med J. 42):473-7; Lam P Y, Breakefield X O., 2001. Potential of gene therapy for brain tumors. Hum Mol. Genet. 10:777-87; Mizuno M, Yoshida J., 2001. Perspectives on postgenome medicine: Gene therapy for brain tumors. Nippon Rinsho. 59:76-80; Bansal K, Engelhard H H., 2000. Gene therapy for brain tumors. Curr Oncol Rep. 2:463-72; Okamoto K. et al., 2000. Gene therapy for brain tumors. No To Shinkei. 52:901-7; Engelhard H H., 2000. Gene therapy for brain tumors: the fundamentals. Surg Neurol. 54:3-9; Jacobs A. et al., 1999. HSV-1-based vectors for gene therapy of neurological diseases and brain tumors: part II. Vector systems and applications. Neoplasia. 1:402-16; Jacobs A. et al., 1999. HSV-1-based vectors for gene therapy of neurological diseases and brain tumors: part I. HSV-1 structure, replication and pathogenesis. Neoplasia 1:387-401; Lowenstein P R. et al., 1999. The basic science of brain-tumour gene therapy. Biochem Soc Trans. 27:873-81; Sandmair A M. et al., 2000. Adenovirus mediated herpes simplex thymidine kinase gene therapy for brain tumors. Adv Exp Med Biol. 465:163-70; Okada H., 1999. Neurosurgery and molecular biology: (series 10) gene therapy and biological therapy for brain tumors. No Shinkei Geka. 27:9-17; Fulci G. et al., 1997. Gene therapy for pediatric brain tumors. Semin Pediatr Neurol. 4:333-9; Mizuno M, Yoshida J., 1997. Gene therapy for brain tumors. Nippon Rinsho. 55:1853-60; Kramm C M. et al., 1995. Gene therapy for brain tumors. Brain Pathol. 5:345-81; Mineta T. et al., 1995. Gene therapy for brain tumors: experimental treatment of malignant brain tumors using recombinant herpes simplex virus type 1 (series 7). No Shinkei Geka. 23:285-92; Shimizu K, Hayakawa T., 1995. Gene therapy for brain tumors: gene therapy for malignant brain tumors using a retroviral vector accompanied by brain-specific expression of genes. No Shinkei Geka. 23:189-94).

Thus, suitable systems for achieving delivery of a nucleic acid construct of the present invention include viral and non-viral delivery systems.

Non-viral systems for achieving delivery of a polynucleotide to pathological cells of the subject include lipid-based nucleic acid delivery systems, such as, for example, those based on lipids such as DOTMA, DOPE, and DC-Chol (Tonkinson et al., 1996. Cancer Investigation 14:54-65). Non-viral delivery systems include those based on cationic lipids, polylysine, dendrimers, and the like. Delivery may also be effected, for example, via nanoparticle bombardment of targeted pathological cells.

A preferred approach for genetically modifying pathological cells of the present invention to express a therapeutic agent of the present invention is by using a viral vector. Viral vectors offer several advantages including higher efficiency of transformation, and targeting to, and propagation in, specific cell types. Viral vectors can also be modified with specific receptors or ligands to alter target specificity through specific cell receptors, such as cancer cell receptors.

Retroviral vectors represent one class of vectors suitable for use with the present invention. Defective retroviruses are routinely used in transfer of genes into mammalian cells (for review see Miller, A. D., Blood 76: 271 (1990)]. A recombinant retrovirus including a nucleic acid sequence encoding a therapeutic agent of the present invention can be constructed using well known molecular techniques. Portions of the retroviral genome can be removed to render the retrovirus replication defective and the replication defective retrovirus can then packaged into virions, which can be used to infect target cells through the use of a helper virus and while employing standard techniques. Protocols for producing recombinant retroviruses and for infecting cells in-vitro or in-vivo with such viruses can be found in, for example, in the references listed in the introduction to the Examples section below. Retroviruses have been used to introduce a variety of genes into many different cell types, including tumor cells of neural origin, epithelial cells endothelial cells, lymphocytes, myoblasts, hepatocytes and bone marrow cells.

Another suitable expression vector is an adenovirus vector. The adenovirus is an extensively studied and routinely used gene transfer vector. Key advantages of an adenovirus vector include relatively high transduction efficiency of dividing and quiescent cells, natural tropism to a wide range of epithelial tissues, where desired, and easy production of high titers [Russel, W. C. [J. Gen. Virol. 81: 57-63 (2000)]. The adenovirus DNA is transported to the nucleus, but does not integrate thereinto. Thus the risk of mutagenesis with adenoviral vectors is minimized, while short term expression is particularly suitable for treating cancer cells. Adenoviral vectors used in experimental cancer treatments are described by Seth et al. [Adenoviral vectors for cancer gene therapy. In: P. Seth (ed.) Adenoviruses: Basic biology to Gene Therapy, Landes, Austin, Tex., (1999) pp. 103-120].

A suitable viral expression vector may also be a chimeric adenovirus/retrovirus vector which combines retroviral and adenoviral components. Preliminary results using such vectors to transduce tumor cells suggest that this new type of viral expression vector is more efficient than traditional expression vectors [Pan et al., Cancer Letters 184: 179-188 (2002)].

The nucleic acid construct may comprise any of various suitable transcription control sequences for achieving a desired tissue type- and/or developmental stage-specific expression of a nucleic acid sequence encoding a therapeutic agent of the present invention. Suitable transcription control sequences for achieving transgene expression in a desired cell type, such as in a tumor cell of neural origin, are well known and routinely employed in the art, as described hereinabove.

It will be appreciated that various construct schemes can be utilized to express two or more therapeutic agents of the present invention, such as an active Karet portion and TrkA, from a single nucleic acid construct. For example, two distinct transcripts can be co-transcribed as a polycistronic message from a single promoter sequence of the nucleic acid construct. To enable co-translation of multiple polypeptides from a single polycistronic message, nucleic acid sequences encoding the polypeptides can be transcriptionally fused via a linker sequence including an internal ribosome entry site (IRES) sequence which enables the translation of the nucleic acid sequence downstream of the IRES sequence. In this case, a transcribed polycistronic RNA molecule including the coding sequences of both polypeptides will be translated from both the capped 5′ end and the internal IRES sequence of the polycistronic RNA molecule to thereby produce both polypeptides. Alternatively, the nucleic acid construct of the present invention can comprise two promoter sequences each being for separately expressing nucleic acid sequences encoding distinct therapeutic agents of the present invention.

A nucleic acid construct of the present invention may be administered to a subject of the present invention according to any one of various suitable routes and administration regimens, depending on the application and purpose. One of ordinary skill in the art, preferably a physician, more preferably a physician specialized in the disease to be treated, will possess the necessary skill for administering a nucleic acid construct of the present invention so as to achieve optimal treatment of the disease in the subject according to the teachings of the present invention. For example, a neurosurgeon will possess the necessary expertise for treating a brain tumor according to the teachings of the present invention. Where possible, when employing a viral vector to achieve delivery of the nucleic acid construct, the vector will be preferably administered directly to the pathological cells, e.g. intratumorally, so as to achieve optimally rapid, safe and effective killing of the pathological cells. However, viral vectors may be selected/constructed capable of specifically targeting the pathological cells so as to render systemic administration feasible. Administration of viral vectors can also be performed by, for example, intravenous or subcutaneous injection into the subject. Following injection, such viral vectors will circulate until they specifically recognize and infect the pathological cells.

The method of the present invention may be practiced by administering to the subject any of various compounds capable of potentiating the cell killing effect resulting from inducing a functional interaction between an active Karet portion of the present invention and TrkA. In particular, the method may comprise administering to the subject nerve growth factor (NGF). A suitable dose of NGF is selected from a dose achieving an NGF concentration at a site of pathological cells of about 5 to about 500 ng/ml, more preferably of about 10 to about 250 ng/ml, more preferably of about 25 to about 100 ng/ml, more preferably of about 40 to about 60 ng/ml, and most preferably of about 50 ng/ml. As is described and illustrated in Example 1 of the Examples section which follows (e.g. FIG. 5e), treatment of pathological cells with 50 ng/ml NGF in conjunction with inducing a functional interaction between an active Karet portion of the present invention and TrkA may be employed to enhance the efficacy of cell killing induced by such interaction.

As used herein the term “about” refers to ±10 percent.

As described hereinabove, the present invention is especially suitable for treatment of cancers, such as cancers of neural origin, and in particular for treatment of brain tumors.

Preferably, cancers which are optimally amenable to treatment via the method of the present invention are characterized by cancer cells which express/comprise TrkA. Expression of TrkA is known in the art to be a feature of primitive neuroectodermal tumor (PNET)/Ewing's sarcoma with neural differentiation (Nogueira E. et al., 1997. Activation of TRK genes in Ewing's sarcoma. Trk A receptor expression linked to neural differentiation. Diagn Mol. Pathol. 6:10-6). As is described and illustrated in Example 1 of the Examples section which follows, the method of the present invention can be used to effectively kill tumor cells of neural origin by inducing therein a functional interaction between an active Karet portion of the present invention and TrkA. As is further described and illustrated in the Examples section which follows, various medulloblastoma and neuroblastoma cell lines were found to display endogenous Karet expression, and medulloblastoma cells were found to display a physical interaction between Karet and TrkA. As is yet further described and illustrated in Example 1 of the Examples section below, analysis of clinical cases of human neuroblastoma showed for the first time a linkage between Karet expression and positive clinical outcome, and between Karet expression and level of differentiation, itself a positive prognostic indicator. As such, it will be appreciated that inducing a functional interaction between an active Karet portion of the present invention and TrkA according to the novel teachings of the present invention can be used to effectively kill cells of human tumors of neural origin such as medulloblastoma and neuroblastoma.

Thus, the method of the present invention is particularly suitable for treating any of various diseases associated with cellular hyperproliferation/abnormally low apoptosis, preferably a cancer, and more preferably a primitive neuroectodermal tumor (PNET)/Ewing's sarcoma such as medulloblastoma or neuroblastoma. Further examples of PNETs/Ewing's sarcoma amenable to treatment via the method of the present invention include, without limitation, ependymoma, choroid plexus papillomas, astrocytoma, retinoblastoma, pineoblastoma, medulloepithelioma, primary rhabdomyosarcoma, atypical teratoid/rhabdoid tumor, pheochromocytoma, Schwanoma, and the like. The method is further suitable for treatment of tumors of neural origin such as glioma.

Preferably, cancers which are optimally amenable to treatment via the method of the present invention are characterized by cancer cells which express/comprise EGFR.

Non-limiting examples of EGFR-associated cancers which can be treated by the method of the present invention include head and neck tumors (head cancer and neck cancer) and lung cancer. The agent(s) of the present invention (e.g., CCM2, TrkA, EGFR polypeptides or the nucleic acid sequences encoding same) can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agents of the present invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, transmucosal, especially trans-nasal, subcutaneous and intramedullary injections, intravenous, or intranasal injections.

Conventional approaches for drug delivery to the central nervous system include: neurosurgical strategies (e.g., intracerebral injection or intracerebroventricular infusion); molecular manipulation of the agent (e.g., use of immunoliposomes directed to CNS), pharmacological strategies designed to increase the lipid solubility of an agent (e.g., conjugation of water-soluble agents to lipid or cholesterol carriers); and the transitory disruption of the integrity of the blood brain barrier (BBB) by hyperosmotic disruption (resulting from the infusion of a mannitol solution into the carotid artery or the use of a biologically active agent such as an angiotensin peptide).

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (the agent of the present invention, e.g., the CCM2, TrkA or EGFR polypeptide or nucleic acid sequence) effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to induce cell death (apoptosis) (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as it is further detailed above.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578, 3,853,987; 3,867,517, 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### General Materials and Experimental Methods

Viability of cells was evaluated using the Live/dead viability/cytotoxicity kit (Cat. No. L-3224), Molecular Probes, Inc., Eugene, Oreg., USA, according to manufacturer's instructions.

Cell proliferation was evaluated using the Cell proliferation assay with XTT reagent (Cat. No. 20-300-1000), Biological Industries, Beth Haemek, Israel, according to manufacturer's instructions.

Generation of Constructs

Ha Karet—a full-length mouse Karet (encoding the amino acid sequence of GenBank Accession No. AAR29082.1) was cloned into pcDNA3 (Invitrogen) between EcoRI and XhoI restriction sites, N-terminally haemagglutintin tag (Ha) was cloned upstream to Karet between BamHI and EcoRI restriction sites.

Ha PTB—PTB domain (693 bp) of mouse Karet was cloned into pcDNA3 between EcoRI and XhoI restriction sites, N-terminally hemagglutinin tag (Ha) was cloned upstream to the PTB between BamHI and EcoRI restriction sites.

Ha non PTB—C terminal of mouse Karet (non-PTB, 680 bp) was cloned into pcDNA3 between EcoRI and NotI restriction sites, N-terminally hemagglutinin tag (Ha) was cloned upstream to the non PTB between BamHI and EcoRI restriction sites.

Ha Karet F217V—a full-length mouse Karet was cloned into pcDNA3 between EcoRI and XhoI restriction sites. Phenyl alanine at position 217 was point mutated to valine. N-terminally hemagglutinin tag (Ha) was cloned upstream to Karet between BamHI and EcoRI restriction sites.

Ha PTB F217V—PTB domain (693 bp) of mouse Karet was cloned into pcDNA3 between EcoRI and XhoI restriction sites. Phenyl alanine at position 217 was point mutated to valine. N-terminally hemagglutinin tag (Ha) was cloned upstream to the PTB between BamHI and EcoRI restriction sites.

For myristylation a nucleic acid sequence encoding a myristylation sequence MGSSKSKPKDPSQRREF (SEQ ID NO: 11) was used.

Myr Karet—a full-length mouse Karet was cloned into pcDNA3 between EcoRI and XhoI restriction sites, N-terminally myristylation sequence was cloned upstream to Karet between BamHI and EcoRI restriction sites.

Myr Ha PTB⁻-PTB domain (693 bp) of mouse Karet was cloned into pcDNA3 between EcoRI and XhoI restriction sites, N-terminally myristylation sequence was cloned upstream to the PTB between BamHI and EcoRI restriction sites.

Myr Ha non PTB—C terminal of mouse Karet (non-PTB, 680 bp) was cloned into pcDNA3 between EcoRI and NotI restriction sites, N-terminally myristylation sequence was cloned upstream to the non PTB between BamHI and EcoRI restriction sites.

### Example 1

### Karet Induces Tumor Cell Apoptosis

Introduction: Malignant diseases, such as tumors of neural origin, include various highly debilitating and/or lethal diseases, such as medulloblastoma, for which no optimal therapy exists. While reducing the present invention to practice, the present inventors have identified an executor molecule of cell death, as follows.

Experimental Results:

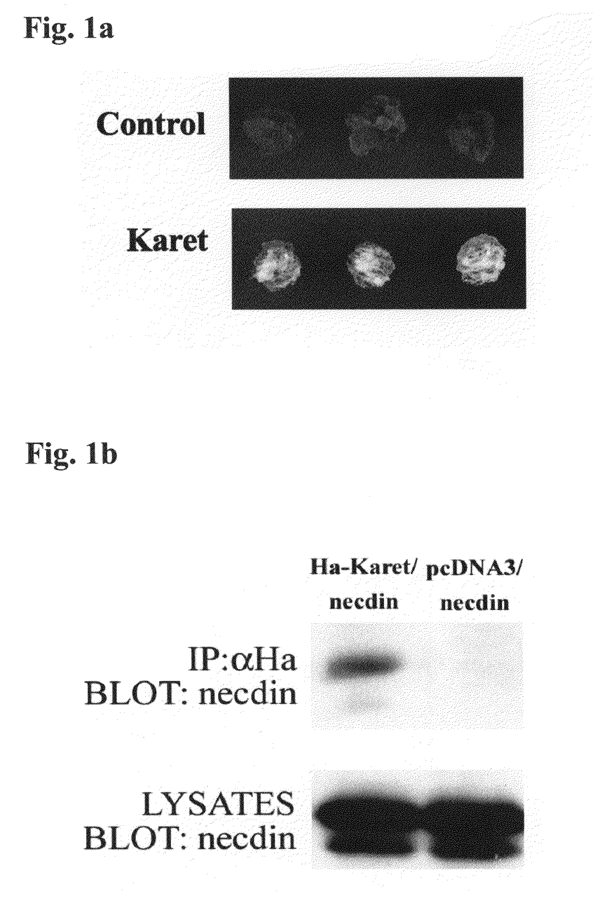

Cloning of Karet as a necdin interactor: To study the intracellular signaling of necdin, a mouse embryonic head Ras Rescue System (RRS) library was screened against necdin as a bait, resulting in identification of a number of potential interactors. One of the positive clones encoded for a mouse protein containing a phosphotyrosine binding domain (PTB). FIG. 1a shows growth of yeast cdc25 strain after transformation with necdin and the PTB domain-containing protein. The interaction was further confirmed by transfection and co-immunoprecipitation studies in mammalian COS cells (FIG. 1b).

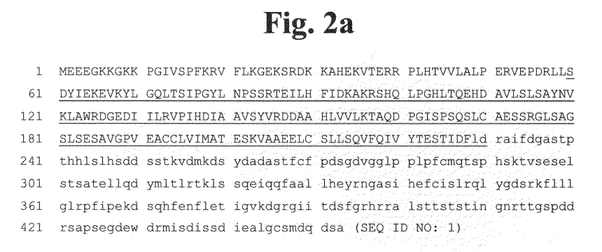

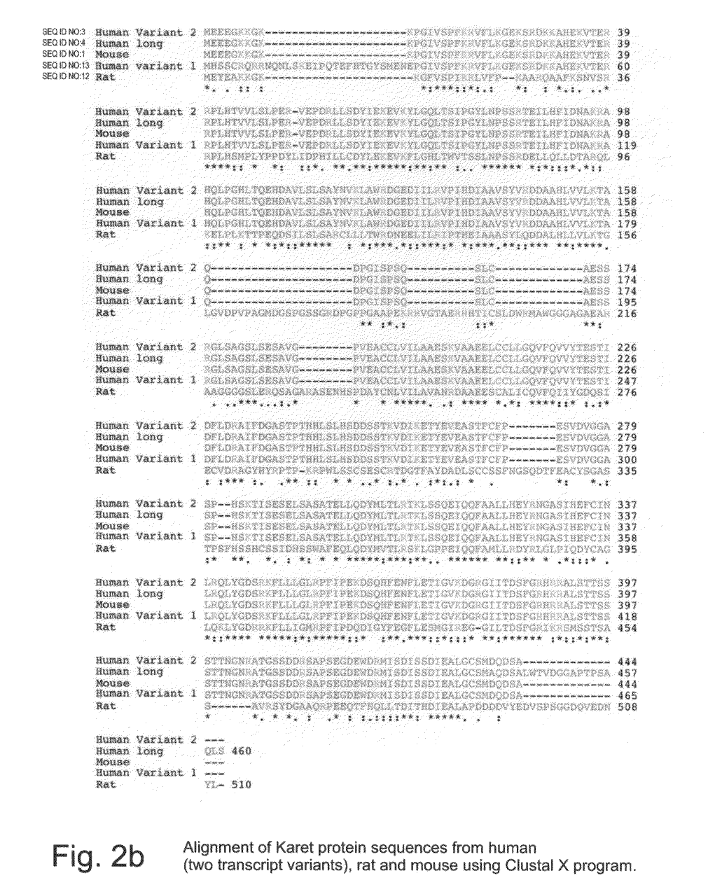

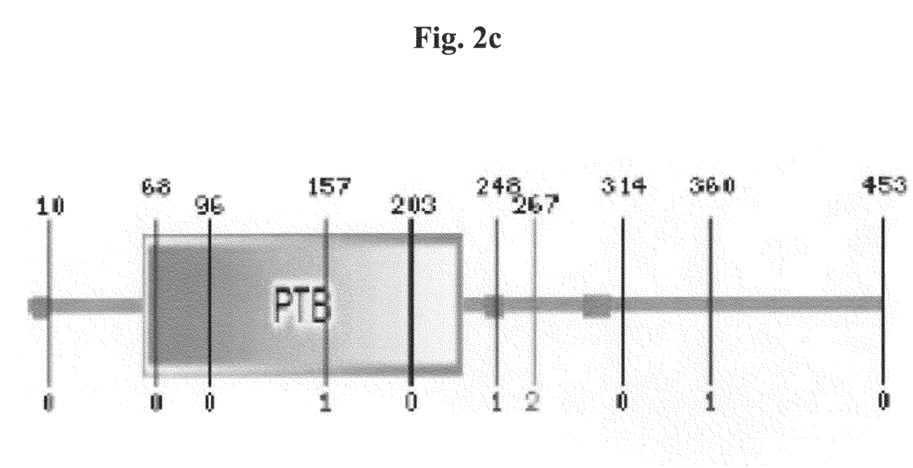

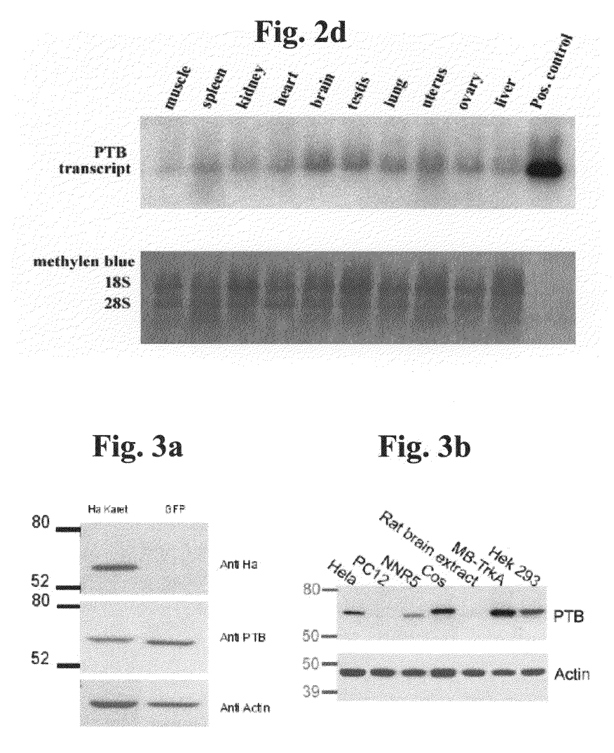

Initial experiments by expression of the PTB domain-containing protein in the PC12-6.24 cell line (based on PC12 cells, a rat neuronal/pheochromocytoma cell line) which overexpresses human TrkA (the production of the cell line is described in Hempstead B L. et al., 1992. Neuron 9:883-896) revealed extensive cell death. The PTB domain-containing protein was accordingly named Karet. Karet sequence analysis placed the 453 amino acid residue protein in the group of Shc-related PTB domain proteins. FIG. 2a shows the amino acid sequence of mouse Karet (SEQ ID NO: 1), and indicates the amino acid sequence of the PTB domain of mouse Karet (determined using SMART software), corresponding to amino acid residues 60-230 (SEQ ID NO: 2). Literature and GenBank scans reveal that the first publication of the Karet sequence was part of a genome sequencing study by the NIH describing 15,000 human and mouse genes (Strausberg et al., 2002) in which Karet is described as MGC4607 (Mammalian Gene Collection # 4607; MGC4607). A subsequent study localized the MGC4607 gene to human chromosome 7 (Scherer et al., 2003). Two papers at the end of 2003 and beginning of 2004 (Denier et al., 2004; Liquori et al., 2003) independently identified MGC4607 (Mammalian Gene Collection # 4607) as the causative agent in Type 2 Cerebral Cavernous Malformation (CCM2) disorder, which results in seizures, stroke and focal neurological deficits. Cerebral cavernous malformations are sporadically acquired or inherited vascular lesions of the nervous system consisting of clusters of dilated thin-walled blood vessels (“caverns”), which have the capacity to hemorrhage, resulting in seizures, stroke and focal neurological deficits (Kondziolka, D. J Neurosurg, 1995. 83 (5): p. 820-4; Gil-Nagel, A., et al. Epilepsy Res, 1995. 21 (1): p. 27-36; Zabramski, J. M., et al. J Neurosurg, 1994. 80 (3): p. 422-32). The physiological importance of Karet was highlighted by recent publications (Uhlik, M. T., et al. Nat Cell Biol, 2003. 5 (12): p. 1104-10; Zawistowski, J. S., et al. Hum Mol Genet, 2005. 14 (17): p. 2521-31), showing that mutations in the PTB domain of Karet, malcavernin and CCM2 are responsible for the cerebral cavernous malformation (CCM) disorder (Liquori, C. L., et al. Am J Hum Genet, 2003. 73 (6): p. 1459-64). There are also mutations in the rest of the protein that also cause CCM2, by creating truncated protein. The murine ortholog of Karet, which was denoted osmosensing scaffold for MEKK3 (OSM), was suggested to be a scaffold for a Rac1/MEKK3/MKK3 signaling complex regulating p38 MAP kinase activation (Uhlik, M. T., et al. Nat Cell Biol, 2003. 5 (12): p. 1104-10). Although the function of Karet/CCM2 is not elucidated, Karet was shown to interact via its PTB domain with CCM1 (KRIT1), a modulator of integrin-mediated cell-cell or cell-matrix adhesion (Zawistowski, J. S., et al. Hum Mol Genet, 2005. 14 (17): p. 2521-31; Sahoo, T., et al. Hum Mol Genet, 1999. 8 (12): p. 2325-33; Zawistowski, J. S., et al. Hum Mol Genet, 2002. 11 (4): p. 389-96; Denier, C., et al. Ann Neurol, 2004. 55 (2): p. 213-20; Plummer, N. W., et al. Am J Pathol, 2004. 165 (5): p. 1509-18).

Blast alignment of the mouse sequence (GenBank Accession No. AAR29082.1; SEQ ID NO:1) against the available database showed that the protein is highly conserved between mouse, rat and human (FIG. 2b), with the amino acid sequence of mouse Karet being 89 percent similar in sequence to a 444 amino acid residue form of human Karet (SEQ ID NO: 3; GenBank Accession No. NP_{—}113631; isoform 2), 89 percent similar in sequence to a longer 460 amino acid residue form of human Karet (SEQ ID NO: 4; GenBank Accession No. AAQ15228), and 86.7 percent similar to a 465 amino acids long human Karet isoform 1 (SEQ ID NO:13; GenBank Accession No. NP_{—}001025006.1) according to the standard protein-protein BLAST [blastp] software of the NCBI. The PTB domain of the 444 amino acid residue form of human Karet (SEQ ID NO:3) is located at position 60-230 (SEQ ID NO:5), the PTB domain of the 460 amino acid residue form of human Karet (SEQ ID NO:4) is located at position 60-230 (SEQ ID NO:6), and the PTB domain of the 465 amino acid residue form of human Karet (SEQ ID NO: 13) is located at position 81-251 (SEQ ID NO: 14).

A schematic diagram of Karet protein indicating the location of the PTB domain and intron positions is shown in FIG. 2c.

Northern blot analysis of mouse tissues showed ubiquitous expression of the protein with higher levels of the transcript in brain (FIG. 2d).

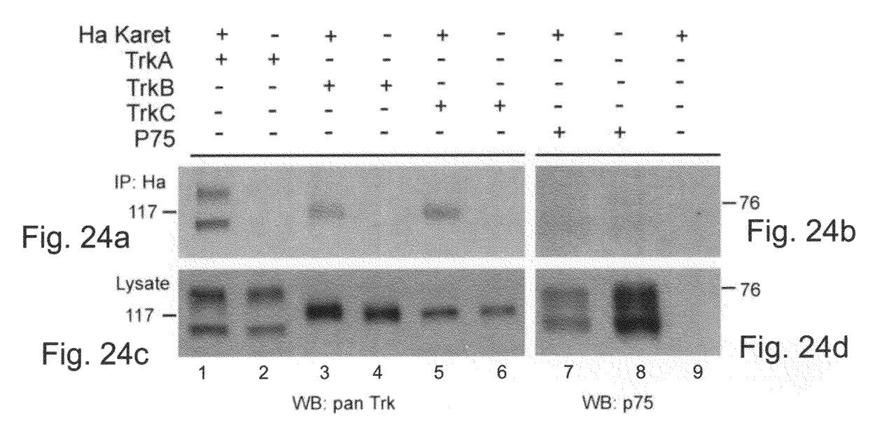

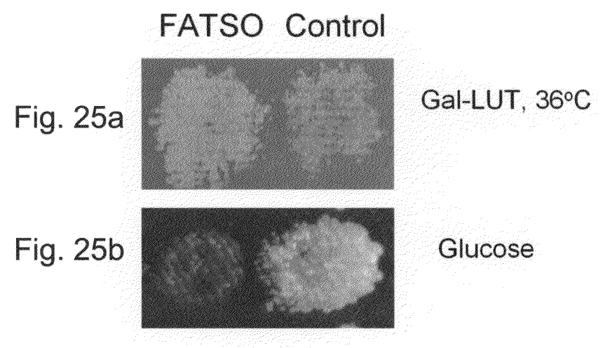

Karet interacts with the neurotrophin receptors TrkA, TrkB or TrkC: The presence of a PTB domain in mouse Karet and its structural homology to the Shc PTB family suggested a possible interaction between Karet and TkrA. With the Help of The Israel Structural Proteomics Center at the Weizmann Institute, a specific antibody against Karet PTB domain was obtained by immunizing rabbits with recombinantly expressed protein. The antibody was shown to recognize overexpressed Karet in Hek293 cells (FIG. 3a) as well as endogenous Karet in different cell lines (FIG. 3b). Co-immunoprecipitation experiments were carried out after transfection of HA-Karet and TrkA expression constructs into COS cells (FIG. 3c) and NNR5 cells, a variant of PC12 lacking TrkA (FIGS. 3d-e). In addition, as is further shown in FIGS. 24a-d, co-immunoprecipitation of Ha Karet with TrkA, TrkB, TrkC or p75 in Hek cells revealed that Karet was co-immunoprecipitated with TrkA, TrkB and TrkC (FIG. 24a) but not with p75 (FIG. 24b). Thus, TrkA was co-immunoprecipitated with Karet from 3 mammalian cell types.

These data suggest interaction of Karet with TrkA, TrkB and TrkC. Further co-immunoprecipitation with TrkA mutants lacking phosphorylation sites at Tyr 490 or Tyr 785 (Tyr to Ala substitution), corresponding to the Shc and PLC binding sites respectively, showed that the protein does not require complete phosphorylation of TrkA for the interaction (FIG. 3f). Since all Trk receptors are conserved on the phosphorylation residues and kinase domain, these observations suggested that Karet may bind the juxtamembrane region of the receptor, which is the less conserved part between different isoforms. Moreover, co-immunoprecipitation analyses in HEK cells revealed co-immunoprecipitation of Karet with EGFR (FIG. 3g)

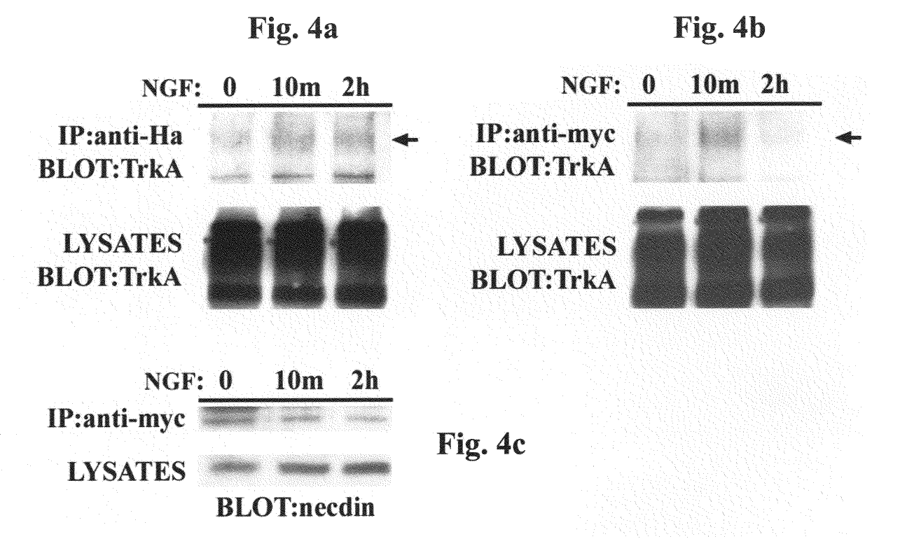

To further examine the interaction of Karet with endogenous TrkA, PC12 cells and necdin-PC12 cells were transfected with mouse Karet by electroporation. Interaction was observed prior to NGF treatment and was enhanced after ligand stimulation for 10 minutes and 2 hours (FIG. 4a). Necdin seems to compete with TrkA for binding to Karet, since in necdin-PC12 cells the interaction between the proteins was not observed after 2 hours of NGF treatment (FIG. 4b), while the interaction between necdin and Karet was robust and constant (FIG. 4c). Surprisingly, the Karet-TrkA interaction was observed in PC12 cells regardless of NGF treatment or starvation of the cells.

Overexpression of Karet induces TrkA-dependent apoptosis of PC12 cells which is increased by NGF treatment: Transient overexpression of mouse Karet in PC12 cells caused approximately 50 percent cell death in the culture, as shown by FACS analysis in FIG. 5a. NGF treatment further increased the death by an additional 30 percent. Live/dead staining (Molecular probes) of PC12 cells expressing Karet showed abundance of dead cells comparing to the control (FIG. 5b-c). Hoechst staining of PC12 cells expressing Karet showed condensed chromatin indicating apoptotic death (FIG. 5d), not observed in control-transfected cells. The presence of necdin in PC12-necdin stable cells attenuated somewhat the apoptotic effects of Karet (FIG. 5e). The effect of Karet on cell survival is TrkA dependent since COS cells and NNR5, a TrkA deficient derivative of PC12 cells, showed no reduction in cell viability and moreover, NNR5 cells co-transfected with Karet and TrkA clones showed about 20 percent reduction in cell survival (FIG. 5f). Thus, transfection of the PTB protein into TrkA-expressing PC12 cells strikingly caused TrkA-dependent NGF-enhanced death of the cells, rather than the expected modulation of differentiation.

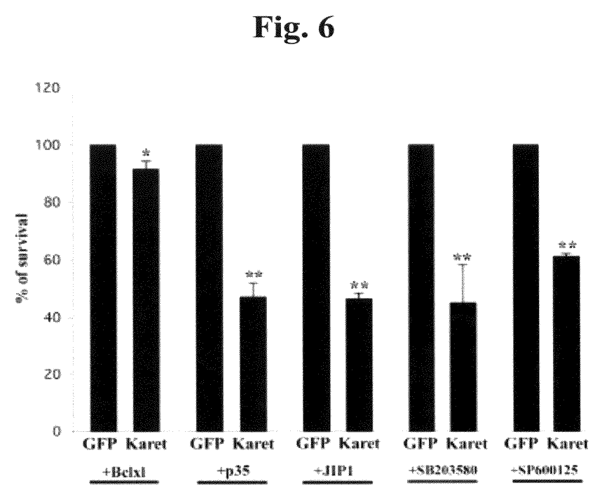

In order to elucidate the mechanism of cell death induced by Karet, the effects of a number of proteins or inhibitors diagnostic for specific pathways were tested. Hoechst staining of PC12 cells expressing Karet and absence of dead cells after treatment with Bclxl, suggested that Karet-induced cell death is a mitochondrial apoptotic pathway (FIG. 6). In contrast p35 (a baculovirus caspase-8 and caspase-10 inhibitor), JIP1 (a Jnk scaffold and inhibitor), SB203580 (a p38 inhibitor), or SB600125 (a broad spectrum inhibitor of stress kinases) did not affect cell death induction by Karet. These results suggest that Karet-induced apoptosis is transduced through the mitochondrial pathway.

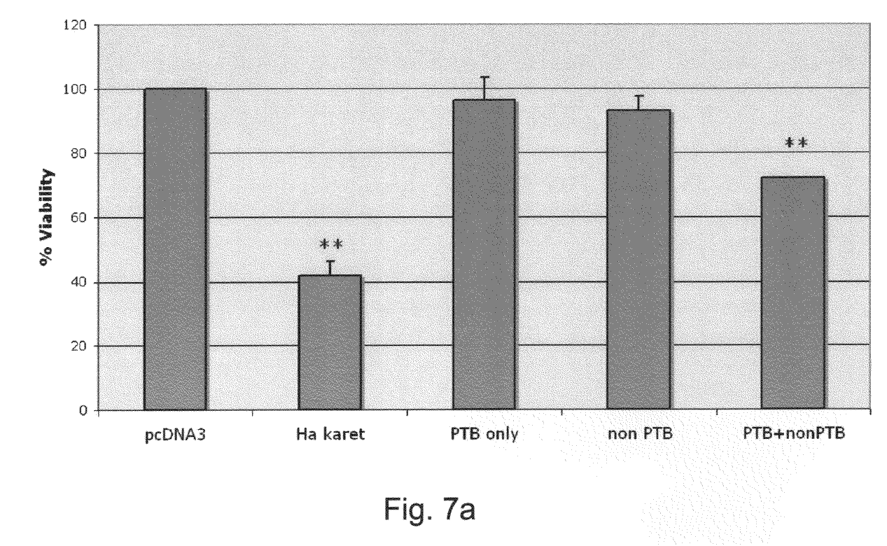

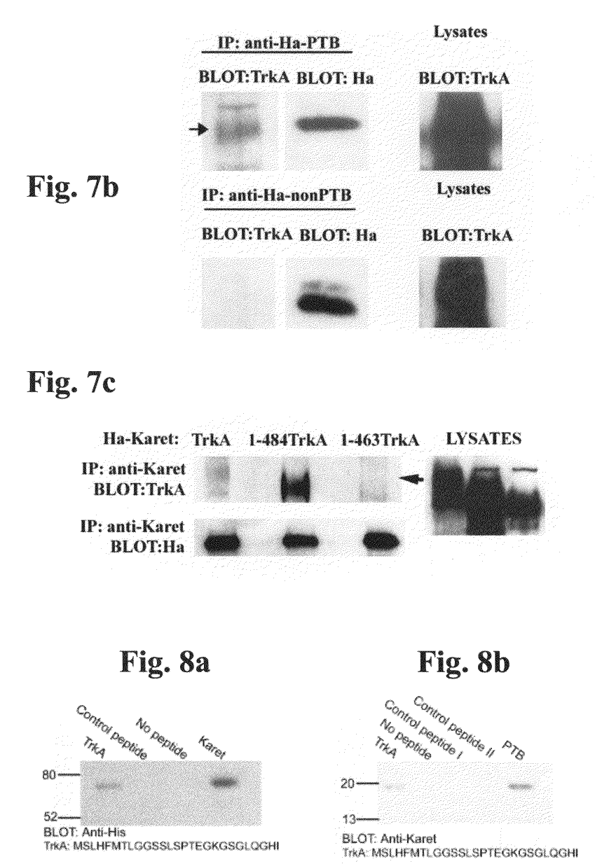

Karet binds a juxtamembrane portion of TrkA polypeptide corresponding to amino acid residues 464-484: The presence of a PTB domain in Karet and its homology to the Shc family suggested that the effect of Karet on cell survival is TrkA dependent, thus the possibility of interaction between TrkA and Karet was investigated. Sequences encoding the N-terminal, PTB-domain-containing, truncation mutant of mouse Karet corresponding to amino acid residues 1-228 [SEQ ID NO: 7; Karet(1-228)], and the C-terminal truncation mutant of the protein corresponding to amino acid residues 228-453 [SEQ ID NO:8; Karet(228-453)] were cloned separately into an HA-tag vector. As is shown in FIG. 7a, expression of Karet caused cell death in PC12 cells and the combination of Karet PTB domain with the non PTB expressed as two constructs restored some of the death effect caused by Karet. Co-immunoprecipitation experiments showed that the Karet(1-228) (PTB) interacts with TrkA whereas the Karet(228-453) (non-PTB) does not (FIG. 7b). The amino acid sequence of mouse Karet(1-228) is 98 percent similar to the corresponding human sequence in humans, according to the standard protein-protein BLAST [blastp] software of the NCBI.

As described above, no influence of a mutation in Tyr490 (Shc binding site) on the binding of Karet to TrkA was observed. The interaction of Karet with a number of TrkA deletion mutants in NNR5 cells was tested, revealing a specific interaction of Karet with an evolutionarily conserved 21 amino acid residue juxtamembrane region of TrkA at position 464-484 and having the sequence TLGGSSLSPTEGKGSGLQGHI (SEQ ID NO:9; FIG. 7c; Peng, X., et al., 1995. Neuron 15:395-406). This portion of TrkA is fully conserved in the human and mouse homologs. The interaction was verified by in-vitro co-precipitation of a biotinylated synthetic TrkA juxtamembrane region fragment 27 residues in length at position 458-484 and having the sequence MSLHFMTLGGSSLSPTEGKGSGLQGHI (SEQ ID NO:10) with recombinant PTB-domain containing Karet(1-228) truncation mutant (FIG. 8a). Additionally, in-vitro precipitation of the PTB domain alone revealed that the PTB domain is responsible for the interaction with TrkA (FIG. 8b).

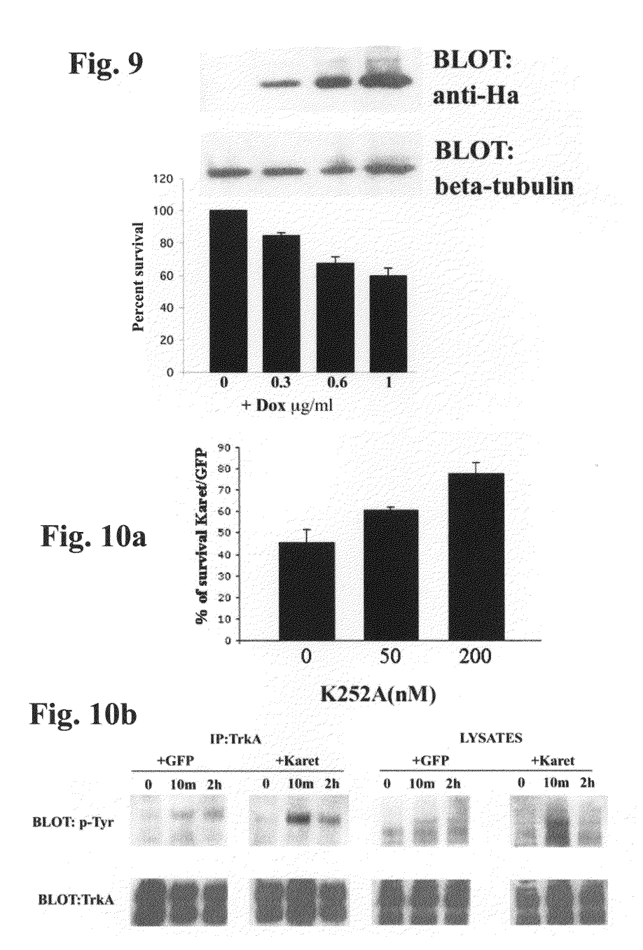

The observations so far suggested that cell death effects are dependent on increased expression of Karet and/or TrkA. This possibility was examined by using the Tet-on system in PC12 to modulate Karet expression. Sequences encoding Karet were cloned in a vector with the inducible promoter and transfected into PC12-tet cells. Cell death in this paradigm was well correlated with expression levels of the Karet protein (FIG. 9).

Karet modulates TrkA phosphorylation: PC12 cells expressing Karet were incubated with K252a, a known inhibitor of TrkA phosphorylation. The presence of this substance in the medium diminished the cell death effect of Karet (FIG. 10a) in a dose-dependent fashion. In order to address this issue further, the levels of Tyr phosphorylation of TrkA of PC12-6.24 cells were examined. Expression of Karet in this cell line significantly enhanced the phosphorylation of the receptor (FIG. 10b).

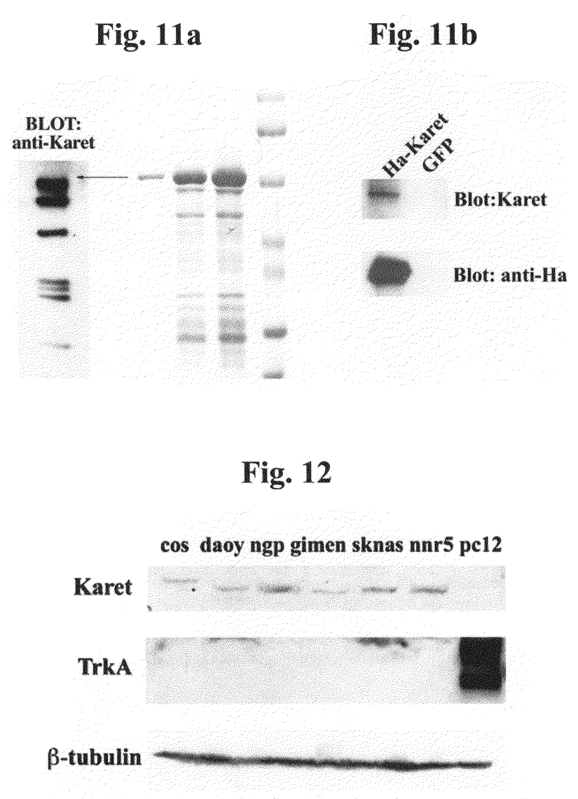

Correlation between Karet expression and improved prognosis in brain tumors: An anti-Karet antibody was produced by immunizing rabbits with recombinant GST-Karet, immunopurified by affinity column purification (FIG. 11a), and verified by recognition of HA-tagged Karet expressed in COS cells (FIG. 11b). A number of neuroblastoma and medulloblastoma cell lines as well as COS, PC12 and NNR5 cells lines were checked for endogenous expression of Karet (FIG. 12). Interestingly, PC12 cells exhibit extremely low levels of endogenous Karet (FIG. 3b), and also the only cell line in this panel with clear expression of TrkA.

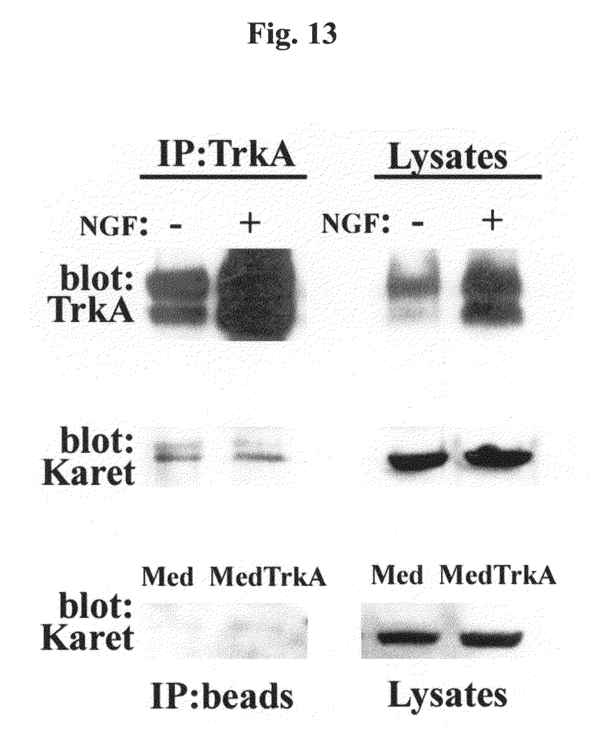

The interaction of Karet with TrkA in pediatric tumor cells was tested in the TrkA-transfected medulloblastoma model (Chou et al., 2000). Karet co-immunoprecipitates with TrkA from the medulloblastoma-TrkA cells with and without NGF treatment (FIG. 13). Interestingly, expression of the TrkA protein is elevated in these cells upon NGF stimulation, suggesting that the Karet-TrkA interaction stabilizes and enhances TrkA protein in pediatric tumor cells.

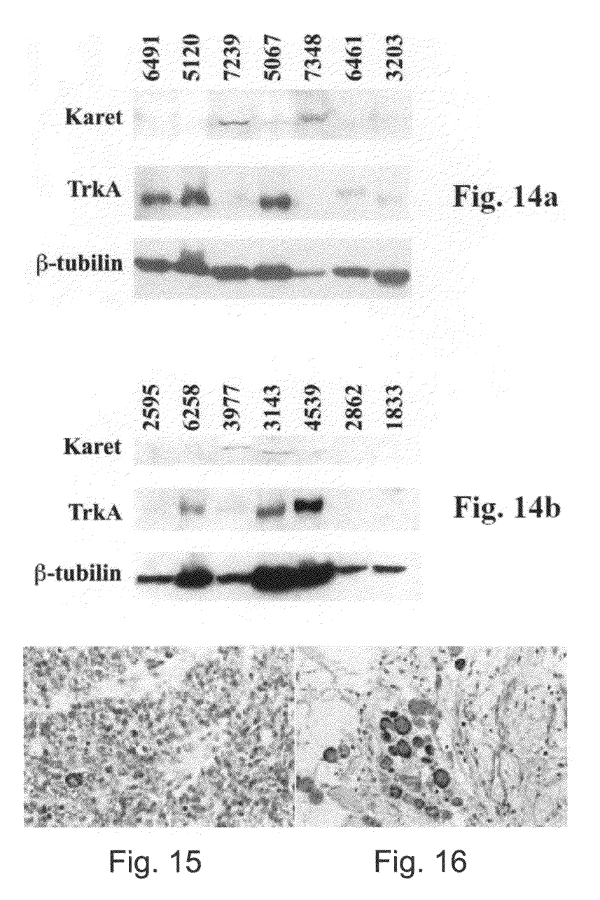

In addition to the medulloblastoma cell line model, clinical samples of neuroblastoma tumors from patients at different stages of the disease were analyzed, in collaboration with the Pediatric Oncology Dept at Schneider Children's Hospital. FIGS. 14a-b show representative Western blot analyses of protein expression levels of Karet and TrkA in neurological tumors from different patients. A total of 38 neuroblastoma samples were examined, of which 12 were classified as early stage disease (Stages 1 and 2) and 26 as late stage disease (Stages 3 and 4). Results showed that 31 of the 38 biopsies were positive for Karet protein, while 18 were positive for both Karet and TrkA. Statistical analysis of the correlation between expression of the two proteins and disease-free survival (5 years from initial diagnosis) revealed a linkage between Karet expression and positive clinical outcome. The cohort of patients with both TrkA and Karet positive tumors enjoyed a positive clinical outcome that was statistically more significant than that observed for TrkA only (in itself already a positive prognostic marker).

An additional examination of tumor material was carried out in collaboration with the Pathology Dept. of Beilinson Hospital, by immunostaining of sections from 15 neuroblastic tumors to characterize Karet expression. FIGS. 15-16, respectively show representative examples of a poorly differentiated neuroblastoma, which is completely Karet-negative, and an intermixed ganglioneuroblastoma (ganglioneuroblastoma represents a mature differentiating tumor with ultimately benign characteristics), which reveals intense and widespread staining of the more differentiated tumor cells. These results depict a correlation of differentiation level/favorable prognosis with increasing Karet expression in human neuroblastic tumors. The Karet-positive cells in the ganglioneuroblastoma samples are mainly those approaching the appearance of mature ganglion cells.

The essence of the presently disclosed findings is that Karet is a pivotal factor in such pathways, and therefore manipulation of Karet expression and/or signaling can be used to target TrkA-expressing tumors, such as TrkA-positive pediatric tumors of neural origin. Moreover, the PTB domain of Karet interacting with TrkA provides a model for death inducing signaling by receptor tyrosine kinases (RTK), and may be used as a template/model for design of molecules that will elicit such effects downstream of any RTK of choice.

### CONCLUSION

The pro-apoptotic effects in tumor cells of Karet, via the interaction of a presently defined N-terminal domain thereof, with a presently defined juxtamembrane domain of TrkA, and the correlation of Karet expression, particularly combined with TrkA expression, with tumor differentiation level/favorable prognosis were unexpectedly uncovered. As such, the presently described findings show that increasing the levels of the aforementioned interaction in cancerous cells can be used to treat tumors, in particular tumors of neural origin.

### Example 2

### Identification of Candidate Polypeptide Interactors of Karet

Experimental Results:

In order to identify candidate polypeptide interactors of Karet, three Ras rescue system screens (RRS) in yeast were performed using the full length Karet protein, the PTB domain or the non-PTB domain as baits with an embryonic mouse brain library (Tcherpakov, M., et al. J Biol Chem, 2002. 277 (51): p. 49101-4). Preliminary results of these screens are presented in Table 1, Table 2 and Table 3, respectively. The Ras rescue system (RRS) method is based on translocation of a mammalian cytoplasmic Ras to the plasma membrane, resulting in activation of a viability pathway in yeast (Broder, Y. C., S. Katz, and A. Aronheim. Curr Biol, 1998. 8 (20): p. 1121-4; Aronheim, A. Methods Mol Biol, 2004. 250: p. 251-62; Hubsman, M., G. et al. Nucleic Acids Res, 2001. 29 (4): p. E18).

TABLE 1
Candidate Karet interactors identified via Ras rescue system in yeast using
whole Karet protein as prey to screen an embryonic mouse brain library

| | | | | | |
|---|---|---|---|---|---|
| | | | | The size of | |
| | | | | the protein | |
| | | | | found relative | |
| | | | | to the whole | Independent |
| Name | Function | Notes | E value | protein/mRNA | repeat |

Cell cycle

| | | | | | |
|---|---|---|---|---|---|
| Cyclin D1, | | | 2e−56 | 123aa/2358bp | 1 |
| mRNA | | | | | |
| Spindlin | A role in cell-cycle | spindlin like protein 2 | 2e−04 | 22aa/2654bp | 1 |
| | regulation during the | is a anti-apoptotic | | | |
| | transition from gamete | protein; | | | |
| | to embryo | overexpression causes | | | |
| | | G2/M arrest | | | |

Mitochondrial proteins

| | | | | | |
|---|---|---|---|---|---|
| Glyceraldehyde-3- | Second phase of | | 4e−132 | 234aa/333aa | 1 |
| phosphate dehydrogenase | glycolysis; first step | | | | |
| NADH dehydrogenase | NADH + ubiquinone = | Mitochondrial | 2e−26 | 90aa/98aa | 1 |
| subunit 4L | NAD⁺ + ubiquinol | | | | |
| cytochrome P450, family | Plays a key role in | | 2e−37 | 78aa/4157bp | 1 |
| 26, subfamily b, | retinoic acid | | | | |
| polypeptide 1 (Cyp26b1), | metabolism | | | | |
| mRNA | | | | | |
| ATP Synthase, H+ | | Nuclear gene | 4e−15 | 56aa/1879bp | 1 |
| transporting mitochondrial | | encoding | | | |
| F1 complex, beta | | mitochondrial protein | | | |
| subunit(Atp5b) | | | | | |

Metabolic pathways

| | | | | | |
|---|---|---|---|---|---|
| SNCB - beta | Protects neurons from | Non amyloid | 7e−31 | 68aa/133aa | 5 |
| synuclein | staurosporine and 6 | component of senile | | | |
| | hydroxy dopamine (6OHDA)- | plaques found in | | | |
| | stimulated caspase activation | Alzheimer's disease. | | | |
| | in ap53-dependent manner. | | | | |
| Fyn- proto oncogene | Implicated in the | SH2 and SH3 domain | 2e−11 | 32aa/3258bp | 1 |
| tyrosine protein | control of cell growth | | | | |
| kinase, mRNA | | | | | |

Cytoskeleton and associated proteins

| | | | | |
|---|---|---|---|---|
| Kinesin family member | Microtubule-binding | 6e−79 | 128aa/9263bp | 1 |
| 21B (Kif21b), | motor protein probably | | | |
| mRNA | involved in neuronal | | | |
| | dendritic transport | | | |
| Cofilin 1, | Controls reversibly | 1e−07 | 24aa/2285bp | 3 |
| non-muscle | actin polymerization | | | |
| (Cfl1), mRNA | and depolymerization | | | |

Modification proteins

| | | | | |
|---|---|---|---|---|
| Neural precursor cell | E3 ubiquitin protein | 0.001 | 20aa/5494bp | 1 |
| expressed, developmentally | ligase | | | |
| down-regulted gene | | | | |
| 4 (Nedd4), mRNA | | | | |
| Ubiquitin-activating | ubiquintin pathway, | 7e−27 | 25aa/3950bp | 1 |
| enzyme E1, Chr X | first step | | | |
| (Ube1x), mRNA | | | | |
| Beta-1,4- | Formation of the lipid- | 2e−120 | 206aa/438aa | 1 |
| mannosyltransferase | linked precursor | | | |
| | oligosaccharide for N- | | | |
| | glycosylation | | | |

Other

| | | | | | |
|---|---|---|---|---|---|
| CAP, adenylate cyclase- | | RIKEN full-length | 3e−24 | 48aa/2554bp | 1 |
| associated protein 1 | | enriched library, | | | |
| | | clone: 9530052J16 | | | |
| Cysteine and glycine- | Could play a role in | | 1e−11 | 32aa/1770bp | 1 |
| rich protein 1 (Csrp1), | neuronal development | | | | |
| mRNA | | | | | |
| T-complex expressed | | clone: M5C1043P11 | 9e−17 | 40aa/7444bp | 1 |
| gene 4 | | | | | |
| Myeloid leukemia | | | 7e−09 | 25aa/1508bp | 1 |
| factor 2 (Mlf2), | | | | | |
| transcript variant1, | | | | | |
| mRNA | | | | | |
| Hypothetical protein | | | 3e−05 | 20aa/1608bp | 1 |
| LOC634129, transcript | | | | | |
| variant 16 | | | | | |
| (LOC634129), mRNA | | | | | |
| Premature mRNA for | | | 9e−09 | 40aa/3792bp | 1 |
| mKIAA1856 protein | | | | | |
| Similar to | | | | 40aa/4963bpbp | |
| CAGL79, mRNA | | | | | |
| Clone RP23-268C9 | | | 2e−6 | 23aa/189539bp | 1 |
| Clone RP23-422H14 | | | 6e−24 | 51aa/138211bp | 1 |
| BAC clone | | | 1e−09 | 32aa/21096161bp | 2 |
| RP23-291B16 | | | | | |
| RIKEN cDNA 9330120H11 | | | 2e−06 | 24aa/3433bpbp | 1 |
| gene (9330120H11Rik), | | | | | |
| mRNA | | | | | |

TABLE 2
Candidate Karet PTB domain interactors identified via Ras rescue system in yeast
using Karet PTB domain as prey to screen an embryonic mouse brain library

| | | | | | |
|---|---|---|---|---|---|
| | | | | The size of | |
| | | | | the protein | |
| | | | | found relative | |
| | | | | to the whole | Independent |
| Name | Function | Notes | E value | protein/mRNA | repeats |

Cell cycle

| | | | | |
|---|---|---|---|---|
| Spindlin | clone: 7420495G10 | 2e−04 | 22aa/2654bp | 1 |

Transcription and translation factors

| | | | | | |
|---|---|---|---|---|---|
| Homeo box C4 | Sequence-specific | | 1e−08 | 28aa/1748bp | 1 |
| (Hoxc4), mRNA | transcription factor | | | | |
| | which is part of a | | | | |
| | developmental | | | | |
| | regulatory system | | | | |
| Heterogeneous nuclear | Transcriptional | Contains RNA | 3e−06 | 25aa/2027bp | 1 |
| ribonucleoprotein A/B | repressor | recognition motifs | | | |
| | | (RRM) | | | |
| Translation elongation | | | | 187/462aa | 1 |
| factor 1 alpha | | | | | |
| Ribosomal protein | | Clone MGC: 7602 | 3e−38 | 78aa/1088bp | 1 |
| L10A, mRNA | | | | | |

Mitochondrial proteins

| | | | | |
|---|---|---|---|---|
| Cytochrome c oxidase | Alignment at different | 8e−05 | 22/439aa | 2 |
| Subunit I (COI) | positions | 5e−05 | 24/514aa | |

Metabolic pathways

| | | | | | |
|---|---|---|---|---|---|
| SNCB- synuclein beta | | Cellular component: | 7e−31 | 68/133aa | 1 |
| | | mouse-mitochondrion/ | | | |
| | | human-cytoplasm | | | |
| Aes- Amino-terminal | Inhibits NF-kappa-B- | Groucho/TLE N-terminal | 1e−66 | 152/197aa | 1 |
| enhancer of split | regulated gene expression. | Q-rich domain. (The N- | | | |
| | May be required for | terminal domain of the | | | |
| | initiation/maintenance | Grouch/TLE co-repressor | | | |
| | of the differentiated state | proteins are involved in | | | |
| | | oligomerisation). | | | |

Cytoskeleton and associated proteins

| | | | | | |
|---|---|---|---|---|---|
| Stathmin-like 3, | Stathmin is involved | MAPK is responsible | 2e−21 | 53aa/932bp | 1 |
| mRNA | in the regulation of | for the phosphorylation | | | |
| | the microtubule | of stathmin in response | | | |
| | | to NGF. | | | |
| Cofilin 1, non-muscle, | | | 2e−04 | 20aa/2285bp | 1 |
| mRNA | | | | | |

Immune system

| | | | | | |
|---|---|---|---|---|---|
| Granulin (Grn) | Possible cytokine-like | Acts as an autocrine | 1e−41 | 74/589aa | 1 |
| | activity. They may play | growth factor for PC | | | |
| | a role in inflammation, | cells. | | | |
| | wound repair, and tissue | | | | |
| | remodeling. | | | | |
| Prothymosin alpha | Immune | | 4e−08 | 47/173aa | 1 |
| CD81 antigen | Regulation of lymphoma cell | | 1e−30 | 98aa/1510bp | 1 |
| | growth, may acts as the | | | | |
| | viral receptor for HCV | | | | |

Other

| | | | | | |
|---|---|---|---|---|---|
| Sdccag331/teashirt 2 | Serologically defined | Subcellular location: | 2e−45 | 127/1098aa | 1 |
| | colon cancer antigen | Nuclear. | | | |
| | 33 like | | | | |
| DnaJ (Hsp40) | Co-chaperone of | CR domain and J | 7e−33 | 67aa/2838bp | 1 |
| homolog, subfamily A, | Hsc70 | domain, membrane | | | |
| member 2 (Dnaja2), | | bound | | | |
| mRNA | | | | | |
| Clone RP23-94H5, | | | 2e−09 | 29aa/239452bp | 1 |
| chromosome 8 | | | | | |
| RNA binding motif | | | 8e−04 | 18aa/3744bp | 1 |
| protein 12 (Rbm12), | | | | | |
| transcript variant 1, | | | | | |
| mRNA | | | | | |

TABLE 3
Candidate Karet non-PTB domain interactors identified via Ras rescue system

| | | | The size of | |
|---|---|---|---|---|
| | | | protein found | |
| | | | relative to the | |
| | | | whole protein/ | Independent |
| Name | Potential Function | E value | mRNA | Repeats |
| * Fatso (Fto) GenBank | Candidate gene involved | 7e−148 | 265aa/502aa | 1 |
| Accession No. NM_011936 | in processes of | | | |
| (SEQ ID NO: 15) - | programmed cell death, | | | |
| nucleic acids; GenBank | craniofacial | | | |
| Accession No. NP_036066 | development, and | | | |
| (SEQ ID NO: 16) - | establishment of left-right | | | |
| amino acids | asymmetry. | | | |

The human homologue of Fatso (Fto) is encoded by the nucleic acid sequence set by GenBank Accession No. NM_{—}001080432 (SEQ ID NO:20), and the Fatso amino acid sequence is of GenBank Accession No. NP_{—}001073901.1 (SEQ ID NO:21).

### FATSO REFERENCES

1. Bailey, P. J., Klos, J. M., Andersson, E., Karlen, M., Kallstrom, M., Ponjavic, J., Muhr, J., Lenhard, B., Sandelin, A. and Ericson, J. A. Global genomic transcriptional code associated with CNS-expressed genes. Exp. Cell Res. 312 (16), 3108-3119 (2006).
2. Watahiki, A., Waki, K., Hayatsu, N., Shiraki, T., Kondo, S., Nakamura, M., Sasaki, D., Arakawa, T., Kawai, J., Harbers, M., Hayashizaki, Y. and Carninci, P. Libraries enriched for alternatively spliced exons reveal splicing patterns in melanocytes and melanomas. Nat. Methods 1 (3), 233-239 (2004).
3. Okazaki, N., Kikuno, R., Ohara, R., Inamoto, S., Koseki, H., Hiraoka, S., Saga, Y., Nagase, T., Ohara, O. and Koga, H. Prediction of the coding sequences of mouse homologues of KIAA gene: III. the complete nucleotide sequences of 500 mouse KIAA-homologous cDNAs identified by screening of terminal sequences of cDNA clones randomly sampled from size-fractionated libraries. DNA Res. 10 (4), 167-180 (2003).
4. Peters, T., Ausmeier, K., Dildrop, R. and Ruther, U. The mouse Fused toes (Ft) mutation is the result of a 1.6-Mb deletion including the entire Iroquois B gene cluster. Mamm. Genome 13 (4), 186-188 (2002).
5. Peters, T., Ausmeier, K. and Ruther, U. Cloning of Fatso (Fto), a novel gene deleted by the Fused toes (Ft) mouse mutation. Mamm. Genome 10 (10), 983-986 (1999).

### Example 3

### Identification of Mammalian Polypeptide Interactors of Karet

As is disclosed in Example 2 above, polypeptide interactors of Karet were identified in yeast cells via RSS using full length Karet, Karet PTB domain or Karet non-PTB domain. The resulting candidates are validated in mammalian cells by co-immunoprecipitation with full length Karet or Karet PTB domain (Tcherpakov, M., et al. J Biol Chem, 2002. 277(51): p. 49101-4; Conticello, S. G., et al. J Biol Chem, 2003. 278(29): p. 26311-4). Mammalian polypeptide interactors of Karet are examined for co-expression in cell types and tissues responsive to Karet, and for the cell death effects in TrkA-Karet expressing cells. Due to technical limitations of the RRS like the difficulty to screen for interactions dependent on transient modifications (such as tyrosine phosphorylation) and in cases of incompletely saturating screens, experiments are carried out in parallel using a proteomic approach to identify the targets of Karet signaling. This is applied by using Karet constructs to pull down Karet-interacting proteins from mammalian cell or tissue extracts. The affinity purified complexes are fractionated on 1D gel and analyzed by mass spectrometry.

### Example 4

### Identification of Biochemical Pathways Activated by Karet

The data disclosed in Example 1 above teaches that cell death through Karet-TrkA interaction is mediated via a mitochondrial pathway (FIG. 6). Hence, by employing pharmacological inhibitors, antagonists and dominant negatives, pathways linking Trk phosphorylation to mitochondrial responses, experiments are performed in order to analyze the effects of Karet on the activation of Ras, Raf, MEK, Erk and Rsk, as well as the Shc-PI3K-Akt pathway, and to analyze the impact of these on Bad, Bcl2 and BcIXI at the mitochondrion, as well as potential p53/p73 mediated effects (Nakagawara, A. Cancer Lett, 2001. 169(2): p. 107-14; Lee, A. F., et al. J Neurosci, 2004. 24(41): p. 9174-84). In order to enable reproducible analysis which is not based on over-expressed proteins, NNR5 cell lines stably transfected with Karet and TrkA under the control of the tet-on system are employed to allow precise titration of Karet and TrkA levels.

### Example 5

### Structure-Function Analysis of the Karet-TrkA Interaction

Experiments are performed in order to enable identification of compounds capable of regulating the Karet-TrkA interaction so as to achieve therapeutic elimination of tumor cells of neural origin. The structural basis for the Karet-TrkA interaction is established, and its specificity is analyzed in a series of point and deletion mutants of TrkA used in co-transfection and co-immunoprecipitation experiments to delineate the binding epitopes for Karet on the receptor. The data disclosed in Example 1 above demonstrates localization of one binding epitope to residues 464-484 in the juxtamembrane region of TrkA (FIG. 8a; Yano, H., et al. J Neurosci, 2001. 21(3): p. RC125; Yano, H. and M. V. Chao. J Neurobiol, 2004. 58(2): p. 244-57). Accordingly, individual and combined tyrosine mutants of TrkA are tested for Karet binding and ability to induce Karet signaling.

Experimental results disclosed in Example 1, above, showed that Karet interacts with TrkA though its PTB domain. Consequently, site-directed mutagenesis of the Karet PTB domain is performed, targeting residues analogous to those previously found to be crucial for binding or function in the related PTB domains in Shc and Numb (Yajnik, V., et al. J Biol Chem, 1996. 271(4): p. 1813-6; Yaich, L., et al. J Biol Chem, 1998. 273(17): p. 10381-8). This knowledge is useful for design of dominant-negative or “super-active” Karet mutants for regulating the Karet-TrkA interaction.

In cooperation with the Israel Structural Proteomics Center at the Weizmann Institute, co-crystallization of Karet with TrkA-derived Karet-binding peptides is used to obtain 3D structure data characterizing the Karet-TrkA interaction. Such 3D structure data is then employed in the computational design of compounds capable of regulating the Karet-TrkA interaction so as to achieve therapeutic elimination of tumor cells of neural origin.

### Example 6

### Karet F217V is Incapable of Inducing Cell Death

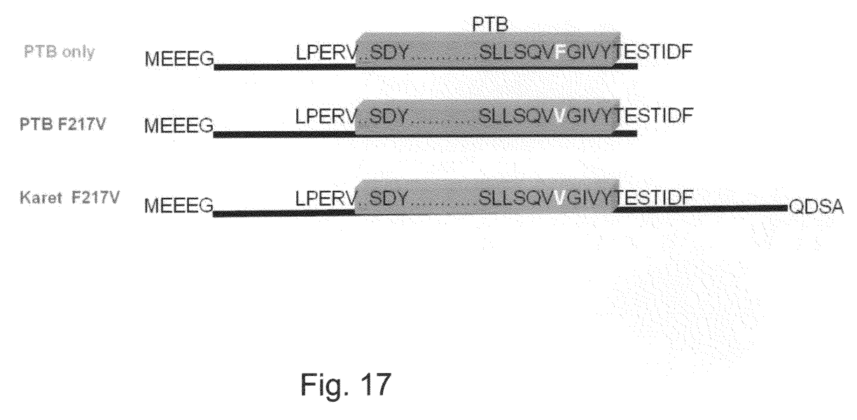

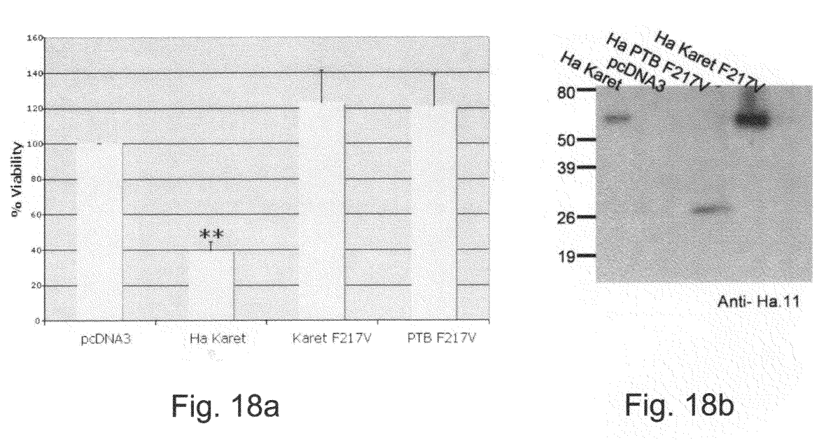

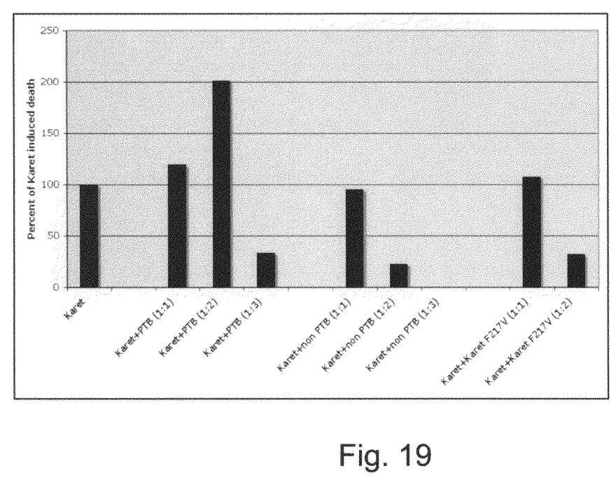

The Karet F217V does not induce cell death—A point mutation phenyl alanine to valine localized in the PTB domain at position 217 (of SEQ ID NO:1; corresponding to position 217 in human Karet SEQ ID NO:3) which is a substitution of phenyl alanine to valine (F217V) was obtained in the full Karet and the PTB domain only construct (FIG. 17). Karet mutants expressed in PC12 cells did not cause cell death as did the wild-type karet protein (FIGS. 18a-b). In addition these mutants and the C terminal domain of the protein (non PTB) seemed to reduce the death in competition assays with Karet, which imply they can act as dominant negative mutants (FIG. 19).

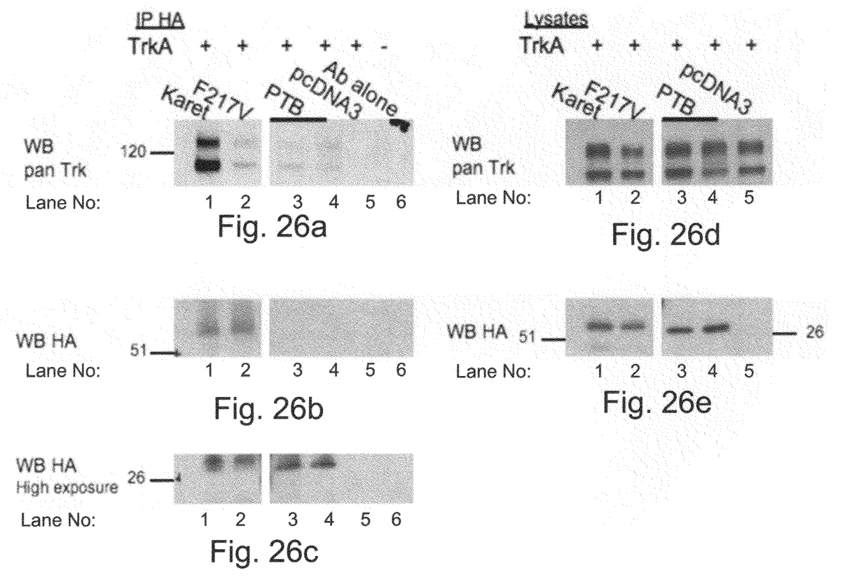

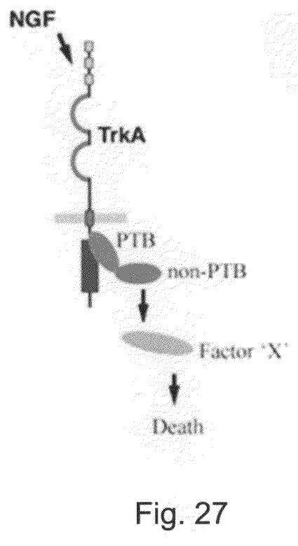

The Karet F217V interacts with TrkA—As is further shown in FIGS. 26a-e, Karet, the F217V Karet mutant, or the PTB domain alone were co-immunoprecipitated in Hek cells with TrkA, demonstrating that the PTB domain of Karet as well as a Karet mutant (F217V) are capable of binding to TrkA.

Thus, these results demonstrate that the reduction in death in the presence of Karet F217V is not a result of lack of interaction between this protein and TrkA.

### Example 7

### Karet Induced Cell Death is TrkA-Dependent

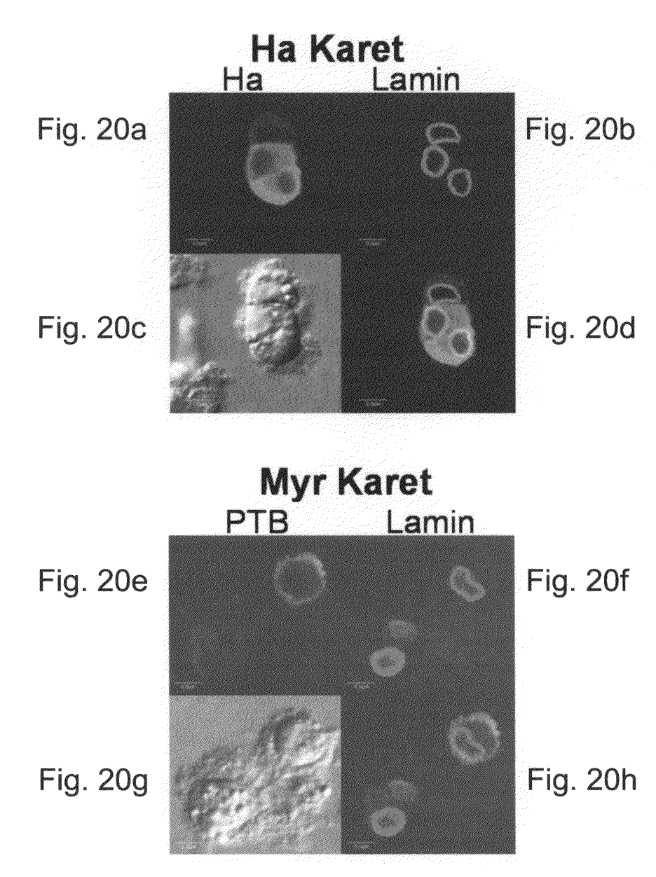

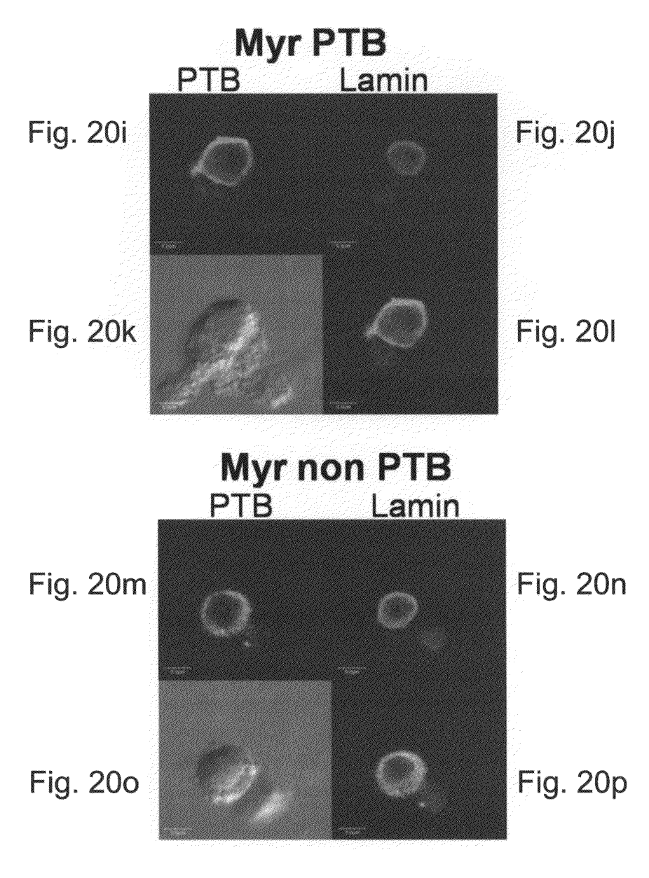

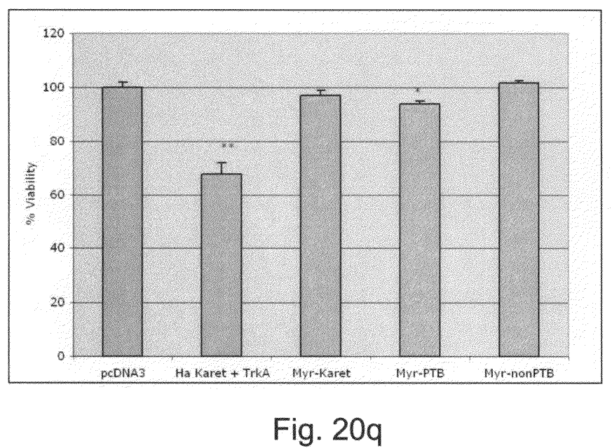

Immunochemistry on different cell lines demonstrated that Karet is predominantly expressed in the cytoplasm (FIG. 20a). In order to verify if the death caused by Karet-TrkA interaction is not an outcome of transporting Karet closer to the membrane, myristylated constructs of Karet, PTB and the non PTB domain were prepared and transfected to PC12 cells or NNR5 cells (which are devoid of TrkA). It was expected that linking an identified Karet-binding epitope to a myristylation membrane-targeting sequence will result in localization of Karet at the plasma membrane.

Experimental Results

As is shown in FIGS. 20a-p, the myristylated constructs directed the expression of Karet to the cell membrane. As is shown in FIG. 20q, the myristylated form of karet was incapable of inducing cell death when transfected alone (without TrkA), as compared to the non-myristylated Karet when transfected with TrkA.

Thus, these results demonstrate that Karet-induced cell death is mediated by and dependent on TrkA.

### Example 8

### Karet-Induces Cell Death Via TrkA or EGFR in Human Cells

To test whether Karet is capable of inducing cell death in other cells such as human HEK or NNR5 cells, the cells were transfected with various receptors and Karet constructs and the effect on cell death was determined by microscopic observation of cell morphology and using cell viability assays, as follows.

Experimental Results

As is shown in FIGS. 21a-q, co-transfection of Karet with TrkA or of Karet with EGFR resulted in morphology change which is consistent with cell death.

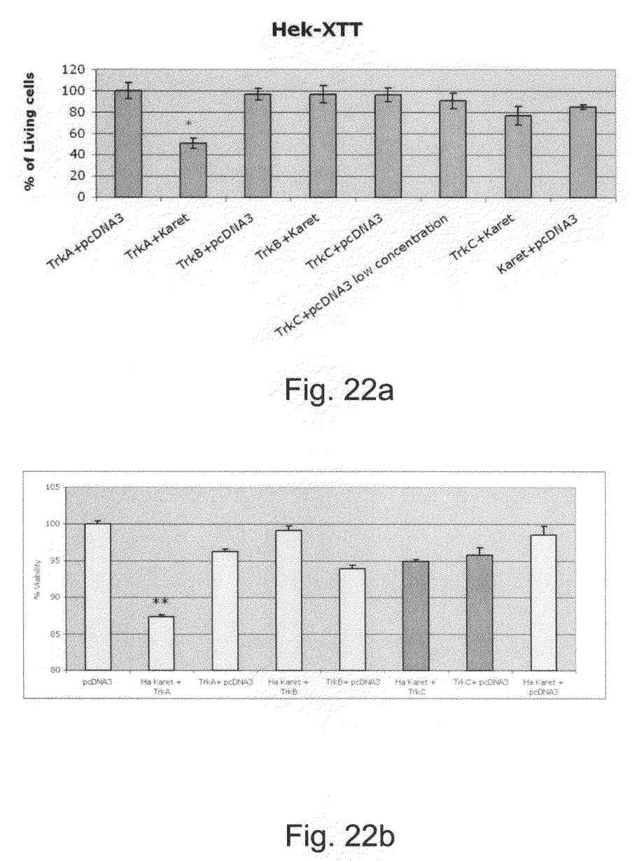

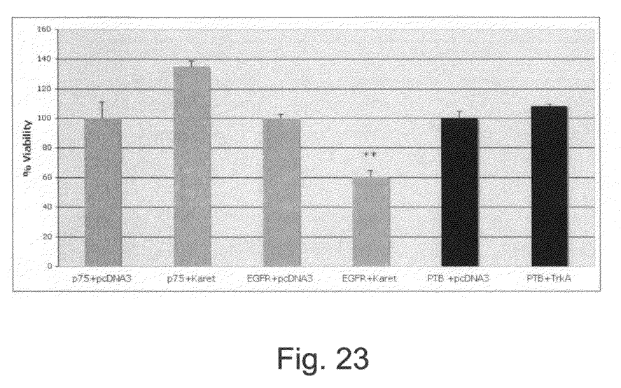

In addition, co-transfection of Karet with TrkA in Hek cells (FIG. 22a) or NNR5 cells (FIG. 22b) resulted in significant cell death [a decrease of 50% in living cells in HEK cells (FIG. 22a) or 13% in viability in NNR5 cells (FIG. 22b)].

Similarly, co-transfection of EGFR and Karet in Hek cells resulted in a significant cell death (a decrease of 40% in cell viability).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, and sequences identified by their GenBank accession numbers mentioned in this specification are herein incorporated in their entirety by reference into the specification, to the same extent as if each individual publication, patent, or sequence identified by its GenBank accession number was specifically and individually indicated to be incorporated herein by reference. In addition, citation or identification of any reference in this application shall not be construed as an admission that such reference is available as prior art to the present invention.

### REFERENCES CITED

### Additional References are Cited in the Text

1. Aronheim, A., Ras signaling pathway for analysis of protein-protein interactions in yeast and mammalian cells. Methods Mol Biol, 2004. 250: p. 251-62.
2. Barker, P. A. (2004). p75NTR is positively promiscuous: novel partners and new insights. Neuron 42, 529-533.
3. Bono, F., Lamarche, I., and Herbert, J. M. (1997). NGF exhibits a pro-apoptotic activity for human vascular smooth muscle cells that is inhibited by TGFbeta1. FEBS Lett 416, 243-246.
4. Broder, Y. C., S. Katz, and A. Aronheim, The ras recruitment system, a novel approach to the study of protein-protein interactions. Curr Biol, 1998. 8 (20): p. 1121-4.
5. Brodeur, G. M. (2003). Neuroblastoma: biological insights into a clinical enigma. Nat Rev Cancer 3, 203-216.
6. Bronfman, F. C., and Fainzilber, M. (2004). Multi-tasking by the p75 neurotrophin receptor: sortilin things out? EMBO Rep 5, 867-871.
7. Brummelkamp, T. R., R. Bernards, and R. Agami, A system for stable expression of short interfering RNAs in mammalian cells. Science, 2002. 296 (5567): p. 550-3.
8. Chou, T. T., Trojanowski, J. Q., and Lee, V. M. (2000). A novel apoptotic pathway induced by nerve growth factor-mediated TrkA activation in medulloblastoma. J Biol Chem 275, 565-570.
9. Chou, T. T., J. Q. Trojanowski, and V. M. Lee, p38 mitogen-activated protein kinase-independent induction of gadd45 expression in nerve growth factor-induced apoptosis in medulloblastomas. J Biol Chem, 2001. 276 (44): p. 41120-7.
10. Conticello, S. G., et al., The prodomain of a secreted hydrophobic mini-protein facilitates its export from the endoplasmic reticulum by hitchhiking on sorting receptors. J Biol Chem, 2003. 278 (29): p. 26311-4.
11. Denier, C., Goutagny, S., Labauge, P., Krivosic, V., Arnoult, M., Cousin, A., Benabid, A. L., Comoy, J., Frerebeau, P., Gilbert, B., et al. (2004). Mutations within the MGC4607 gene cause cerebral cavernous malformations. Am J Hum Genet. 74, 326-337.
12. Denier, C., et al., Clinical features of cerebral cavernous malformations patients with KRIT1 mutations. Ann Neurol, 2004. 55 (2): p. 213-20.
13. Eberhart, C. G., Kaufman, W. E., Tihan, T., and Burger, P. C. (2001). Apoptosis, neuronal maturation, and neurotrophin expression within medulloblastoma nodules. J Neuropathol Exp Neurol 60, 462-469.
14. Gil-Nagel, A., et al., Familial cerebral cavernous angioma: clinical analysis of a family and phenotypic classification. Epilepsy Res, 1995. 21 (1): p. 27-36.
15. Grotzer, M. A., Janss, A. J., Phillips, P. C., and Trojanowski, J. Q. (2000). Neurotrophin receptor TrkC predicts good clinical outcome in medulloblastoma and other primitive neuroectodermal brain tumors. Klin Padiatr 212, 196-199.
16. Huang, E. J., and Reichardt, L. F. (2003). Trk Receptors: Roles in Neuronal Signal Transduction. Annu Rev Biochem 72, 609-642.
17. Hubsman, M., G. Yudkovsky, and A. Aronheim, A novel approach for the identification of protein-protein interaction with integral membrane proteins. Nucleic Acids Res, 2001. 29 (4): p. E18.
18. Katsetos, C. D., Del Valle, L., Legido, A., de Chadarevian, J. P., Perentes, E., and Mork, S. J. (2003). On the neuronal/neuroblastic nature of medulloblastomas: a tribute to Pio del Rio Hortega and Moises Polak. Acta Neuropathol (Berl) 105, 1-13.
19. Kim, J. Y., et al., Activation of neurotrophin-3 receptor TrkC induces apoptosis in medulloblastomas. Cancer Res, 1999. 59 (3): p. 711-9.
20. Kim, J. Y., Nelson, A. L., Algon, S. A., Graves, O., Sturla, L. M., Goumnerova, L. C., Rowitch, D. H., Segal, R. A., and Pomeroy, S. L. (2003). Medulloblastoma tumorigenesis diverges from cerebellar granule cell differentiation in patched heterozygous mice. Dev Biol 263, 50-66.
21. Kondziolka, D., L. D. Lunsford, and J. R. Kestle, The natural history of cerebral cavernous malformations. J Neurosurg, 1995. 83 (5): p. 820-4.
22. Lavoie, J. F., et al., TrkA induces apoptosis of neuroblastoma cells and does so via a p53-dependent mechanism. J Biol Chem, 2005. 280 (32): p. 29199-207.
23. Lee, A. F., et al., Evidence that DeltaNp73 promotes neuronal survival by p53-dependent and p53-independent mechanisms. J Neurosci, 2004. 24 (41): p. 9174-84.
24. Liquori, C. L., Berg, M. J., Siegel, A. M., Huang, E., Zawistowski, J. S., Stoffer, T., Verlaan, D., Balogun, F., Hughes, L., Leedom, T. P., et al. (2003). Mutations in a gene encoding a novel protein containing a phosphotyrosine-binding domain cause type 2 cerebral cavernous malformations. Am J Hum Genet 73, 1459-1464.
25. Lucarelli, E., Kaplan, D., and Thiele, C. J. (1997). Activation of trk-A but not trk-B signal transduction pathway inhibits growth of neuroblastoma cells. Eur J Cancer 33, 2068-2070.
26. Muragaki, Y., Chou, T. T., Kaplan, D. R., Trojanowski, J. Q., and Lee, V. M. (1997). Nerve growth factor induces apoptosis in human medulloblastoma cell lines that express TrkA receptors. J Neurosci 17, 530-542.
27. Nakagawara, A. (2001). Trk receptor tyrosine kinases: a bridge between cancer and neural development. Cancer Lett 169, 107-114.
28. Nakagawara, A., and Ohira, M. (2004). Comprehensive genomics linking between neural development and cancer: neuroblastoma as a model. Cancer Lett 204, 213-224.
29. Ohira, M., et al., A review of DNA microarray analysis of human neuroblastomas. Cancer Lett, 2005. 228 (1-2): p. 5-11.
30. Peng, X., et al., Deletion of a conserved juxtamembrane sequence in Trk abolishes NGF-promoted neuritogenesis. Neuron, 1995.15 (2): p. 395-406.
31. Plummer, N. W., et al., Loss of p53 sensitizes mice with a mutation in Ccm1 (KRIT1) to development of cerebral vascular malformations. Am J Pathol, 2004. 165 (5): p. 1509-18.
32. Pomeroy, S. L., and Sturla, L. M. (2003). Molecular biology of medulloblastoma therapy. Pediatr Neurosurg 39, 299-304.
33. Pomeroy, S. L., Sutton, M. E., Goumnerova, L. C., and Segal, R. A. (1997). Neurotrophins in cerebellar granule cell development and medulloblastoma. J Neurooncol 35, 347-352.
34. Roux, P. P., and Barker, P. A. (2002). Neurotrophin signaling through the p75 neurotrophin receptor. Prog Neurobiol 67, 203-233.
35. Rubin, J. B., and Segal, R. A. (2003). Growth, survival and migration: the Trk to cancer. Cancer Treat Res 115, 1-18.
36. Sahoo, T., et al., Mutations in the gene encoding KRIT1, a Krev-1/rap1a binding protein, cause cerebral cavernous malformations (CCM1). Hum Mol Genet, 1999. 8 (12): p. 2325-33.
37. Scherer, S. W., Cheung, J., MacDonald, J. R., Osborne, L. R., Nakabayashi, K., Herbrick, J. A., Carson, A. R., Parker-Katiraee, L., Skaug, J., Khaja, R., et al. (2003). Human chromosome 7: DNA sequence and biology. Science 300, 767-772.
38. Schramm, A., et al., Biological effects of TrkA and TrkB receptor signaling in neuroblastoma. Cancer Lett, 2005. 228 (1-2): p. 143-53.
39. Segal, R. A. (2003). Selectivity in neurotrophin signaling: theme and variations. Annu Rev Neurosci 26, 299-330.
40. Strausberg, R. L., Feingold, E. A., Grouse, L. H., Derge, J. G., Klausner, R. D., Collins, F. S., Wagner, L., Shenmen, C. M., Schuler, G. D., Altschul, S. F., et al. (2002). Generation and initial analysis of more than 15,000 full-length human and mouse cDNA sequences. Proc Natl Acad Sci USA 99, 16899-16903.
41. Takazaki, R., Nishimura, I., and Yoshikawa, K. (2002). Necdin is required for terminal differentiation and survival of primary dorsal root ganglion neurons. Exp Cell Res 277, 220-232.
42. Tcherpakov, M., Bronfman, F. C., Conticello, S. G., Vaskovsky, A., Levy, Z., Niinobe, M., Yoshikawa, K., Arenas, E., and Fainzilber, M. (2002). The p75 neurotrophin receptor interacts with multiple MAGE proteins. J Biol Chem 277, 49101-49104.
43. Uhlik, M. T., Abell, A. N., Johnson, N. L., Sun, W., Cuevas, B. D., Lobel-Rice, K. E., Horne, E. A., Dell'Acqua, M. L., and Johnson, G. L. (2003). Rac-MEKK3-MKK3 scaffolding for p38 MAPK activation during hyperosmotic shock. Nat Cell Biol 5, 1104-1110.
44. Ullrich, N. J., and Pomeroy, S. L. (2003). Pediatric brain tumors. Neurol Clin 21, 897-913.
45. Woo, C. W., Lucarelli, E., and Thiele, C. J. (2004). NGF activation of TrkA decreases N-myc expression via MAPK path leading to a decrease in neuroblastoma cell number. Oncogene 23, 1522-1530.
46. Yaich, L., et al., Functional analysis of the Numb phosphotyrosine-binding domain using site-directed mutagenesis. J Biol Chem, 1998. 273 (17): p. 10381-8.
47. Yajnik, V., et al., Identification of residues within the SHC phosphotyrosine binding/phosphotyrosine interaction domain crucial for phosphopeptide interaction. J Biol Chem, 1996. 271 (4): p. 1813-6.
48. Yan, C., Liang, Y., Nylander, K. D., and Schor, N. F. (2002). TrkA as a life and death receptor: receptor dose as a mediator of function. Cancer Res 62, 4867-4875.
49. Yano, H., et al., Association of Trk neurotrophin receptors with components of the cytoplasmic dynein motor. J Neurosci, 2001. 21 (3): p. RC125.
50. Yano, H. and M. V. Chao, Mechanisms of neurotrophin receptor vesicular transport. J Neurobiol, 2004. 58 (2): p. 244-57.
51. Zabramski, J. M., et al., The natural history of familial cavernous malformations: results of an ongoing study. J Neurosurg, 1994. 80 (3): p. 422-32.
52. Zawistowski, J. S., et al., KRIT1 association with the integrin-binding protein ICAP-1: a new direction in the elucidation of cerebral cavernous malformations (CCM1) pathogenesis. Hum Mol Genet, 2002. 11 (4): p. 389-96.
53. Zawistowski, J. S., et al., CCM1 and CCM2 protein interactions in cell signaling: implications for cerebral cavernous malformations pathogenesis. Hum Mol Genet, 2005. 14 (17): p. 2521-31.

What is claimed is:

1. A method of regulating apoptosis in a pathological cell population of a subject the method comprising administering to the pathological cell population a therapeutically effective amount of at least one agent capable of modulating an interaction between TrkA and CCM2 or EGFR and CCM2, thereby regulating the apoptosis in the pathological cell population.

2. The method of claim 1, wherein said CCM2 comprises an amino acid sequence selected from the group consisting of SEQ ID NOs:1, 3, 4, 12 and 13.

3. The method of claim 1, wherein said TrkA comprises an amino acid sequence selected from the group consisting of SEQ ID NOs:9 and 10.

4. The method of claim 1, wherein said CCM2 is capable of interacting with said TrkA or said EGFR in the pathological cell population, and of inducing death of said pathological cell population.

5. The method of claim 1, wherein the pathological cell population express said TrkA and/or said EGFR.

6. The method of claim 1, wherein said modulating said interaction is increasing and/or stabilizing said interaction between said TrkA or said EGFR and said CCM2.

7. The method of claim 1, wherein the pathological cell population is associated with cancer.

8-10. (canceled)

11. The method of claim 7, wherein said cancer is a medulloblastoma, a neuroblastoma or a pheochromocytoma.

12-13. (canceled)

14. The method of claim 1, wherein said at least one agent comprises CCM2.

15. The method of claim 14, wherein said CCM2 comprises an amino acid sequence selected from the group consisting of SEQ ID NOs: 1, 3, 4, 12 and 13.

16. The method of claim 1, wherein said at least one agent comprises nerve growth factor.

17. The method of claim 1, wherein said at least one agent comprises TrkA.

18. A method of treating a cancer in a subject in need thereof, the method comprising administering to cancer cells of the subject a therapeutically effective amount of Fatso, thereby treating the cancer in the subject.

19. The method of claim 18, wherein said Fatso comprises an amino acid sequence selected from the group consisting of SEQ ID NOs: 16 and 21.

20-22. (canceled)

23. A pharmaceutical composition comprising as an active ingredient a therapeutically effective amount of CCM2, and a pharmaceutically acceptable carrier.

24. A pharmaceutical composition comprising as an active ingredient a therapeutically effective amount of Fatso, and a pharmaceutically acceptable carrier.

25. The pharmaceutical composition of claim 23, wherein said CCM2 comprises an amino acid sequence selected from the group consisting of SEQ ID NOs:1, 3, 4, 12 and 13.

26. The pharmaceutical composition of claim 24, wherein said Fatso comprises an amino acid sequence selected from the group consisting of SEQ ID NOs: 16 and 21.
